(19) United States
(12) Patent Application Publication
(10) Pub. No.: US 2016/0364535 A1
(43) Pub. Date: Dec. 15, 2016

(54) METHOD AND SYSTEM FOR DETERMINING THIRD PARTY LIABILITY UTILIZING SINGLE OR MULTIPLE DATA SOURCES

(71) Applicant: CERNER INNOVATION, INC., KANSAS CITY, KS (US)

(72) Inventor: BRUCE HOWARD KUSENS, North Miami Beach, FL (US)

(21) Appl. No.: 15/179,415

(22) Filed: Jun. 10, 2016

Related U.S. Application Data
(60) Provisional application No. 62/173,462, filed on Jun. 10, 2015.

(51) Int. Cl. G06F 19/00; G06F 17/30; G06Q 20/10; G06Q 40/08; G06Q 30/06

(52) U.S. Cl. CPC G06F 19/328; G06F 19/322; G06Q 40/08; G06Q 30/0613; G06Q 20/102; G06F 17/30864; G06Q 50/24

(57) ABSTRACT

A computer system consolidates and analyzes information related to a patient to evaluate possible third-party liability for the patient's healthcare expenses. In some instances, multiple sources are searched for different indicators of possible third-party liability and/or for evidence as to whether particular healthcare expenses are subject to third-party liability conditions.

### CROSS-REFERENCE TO RELATED APPLICATIONS

This application claims the benefit of U.S. Provisional Patent Application No. 62/173,462, filed Jun. 10, 2015, which is hereby incorporated by reference in its entirety.

### TECHNICAL FIELD

This disclosure relates generally to systems and methods for identifying possible third-party liability for healthcare costs.

### BACKGROUND

Federal, State, and commercial health insurers require that claims for injuries resulting from third party liability be paid by the liability insurance carrier rather than the health insurer. If a patient is covered by Medicare, healthcare providers are legally obligated to search for insurance that is primary to Medicare. Thus for many patients, a healthcare provider has a legal responsibility to uncover the existence of a third party payer/obligor for a patient's healthcare costs. Furthermore, Medicare reimbursement rates are often lower than those of private insurers. Therefore, hospitals have both legal and financial incentives to identify and bill third party payers. Despite those incentives, uncovering third party liability claims is labor intensive and their resolution can take a significant amount of time. As a result, it is common for providers to bill Medicare even if a potential for third party liability exists. Healthcare providers often prefer the certainty of quicker billing and elimination of investigative costs by billing Medicare rather than searching for a third party payer/obligor. Once billed, Medicare investigates whether a third party payer/obligor exists and recovers payment from the liable insurer. Medicare recovers $6 billion each year from private insurers.

It would be desirable to simplify the process for identifying and billing third party payer/obligors, to provide additional revenue for healthcare providers and save Medicare the unnecessary costs of recovering payment from third parties.

### SUMMARY OF THE DISCLOSURE

This summary is provided as a general overview of selected concepts described in greater detail in the following description and in the attached drawings. This summary is not intended to be used in isolation from the remainder of the disclosure to define or interpret the claims.

By scanning data sources available to healthcare providers, the described systems and methods may allow healthcare providers to recover a higher percentage of healthcare costs. The systems and methods may determine whether there exists a third party who may be liable to reimburse a healthcare provider for a patient's healthcare costs. By preferably using multiple data sources, the system can identify which third parties may be liable for which healthcare costs of an identified patient. The healthcare provider can then recover costs from that previously unidentified third party.

One advantage to healthcare providers in identifying and subsequently billing third parties is these third parties may reimburse providers at a higher rate than alternative payers such as Medicare.

In some aspects, the disclosure relates to a method for determining the existence of a third party payer/obligor. The method may comprise accessing a first set of third party liability information for an identified patient. The first set of third party liability information may be derived from a first electronic data source. The first set of third party liability information may include a first indication. The first indication may correspond to one or more of an indication of an existing third party payer/obligor, an indication of an existing legal or judicial settlement, an indication of an existing promise or agreement by a third party to reimburse the healthcare provider for all or some part of the identified patient's healthcare costs, an indication of a first health condition of the identified patient, or an indication of a first healthcare service provided for the identified patient.

The method may comprise accessing a second set of third party liability information for the identified patient. The second set of third party liability information for the identified patient may be derived from a second source. The second source of third party liability information may include an indication. The indication of the second source of third party liability information may correspond to one or more of an indication of an existing third party payer/obligor, an indication of an existing legal or judicial settlement, an indication of an existing promise or agreement of a third party to reimburse the healthcare provider for all or some part of the identified patient's healthcare costs, an indication of a first health condition of the identified patient, or an indication of a first healthcare service provided for the identified patient.

The method may comprise characterizing one or more items from the first set of third party liability information for the identified patient to yield a first set of characterized items. The method may comprise characterizing one or more items from the second set of third party liability information for the identified patient to yield a second set of characterized items. The method may comprise determining whether any of the items in the first set of characterized items correspond to any of the items in the second set of characterized items. The method may comprise checking for errors, inconsistencies, or improbabilities in the values of any items from the first set of characterized items determined to correspond to an item in the second set of characterized items. The method may comprise resolving any errors, inconsistencies, or improbabilities, or flagging any errors, inconsistencies, or improbabilities that cannot be automatically resolved (i.e., resolved by the system without input from a human user). The method may comprise consolidating the first and second sets of third party information to form a composite set of third party liability information for the identified patient.

The method may comprise processing the composite set of third party liability information for the identified patient to identify potential categories of third party liability. The method may comprise processing the composite set of third party liability information for the identified patient to identify potentially liable third party(s). The method may comprise identifying a threshold for assigning third party liability to one or more of the potentially liable third party(s). The method may comprise comparing the threshold for assigning third party liability to one or more of the potentially liable third party(s) to the composite set of third party liability information for the identified patient.

The method may comprise flagging the file, notifying a healthcare provider, or notifying the patient if the threshold for assigning third party liability to one or more of the potentially liable third party(s) is met. The method may comprise assessing the sufficient of information to assign third party liability if the threshold for assigning third party liability is not met.

Processing the composite set of third party liability information may comprise selecting an item from the composite set of third party liability information, and assigning the item a weight according to a score for reliability of the information. Processing the composite set of third party liability information may comprise identifying potentially liable third party(s) based at least in part on the weighted item. The score for reliability may be based on the source of the information, the completeness of the information, or both. The method may comprise scoring the likelihood of third party payment based on the weight assigned to the information used to identify the third party payment.

In some aspects, this disclosure relates to a system for determining the existence of a third party payer/obligor. The system may comprise one or more interfaces configured to exchange information with at least one of a healthcare provider and a patient. The system may comprise a network over which information may be exchanged between other system components. The system may comprise a data consolidation engine. The data consolidation engine may be configured to access a first set of third party liability information for an identified patient. The data consolidation engine may be configured to access a second set of third party liability information for the identified patient. The data consolidation engine may be configured to characterize one or more items from the first set of third party liability information for the identified patient to yield a first set of characterized items. The data consolidation engine may be configured to characterize one or more items from the second set of third party liability information for the identified patient to yield a second set of characterized items. The data consolidation engine may be configured to determine whether any of the items in the first set of characterized items correspond to any of the items in the second set of characterized items. The data consolidation engine may be configured to consolidate the first and second sets of third party information to form a composite set of third party liability information for the identified patient.

The system may comprise a data integrity engine. The data integrity engine may be configured to check for errors, inconsistencies, or improbabilities in the values of any items from the first set of characterized items determined to correspond to an item in the second set of characterized items. The data integrity engine may be configured to resolve any errors, inconsistencies, or improbabilities or flag any errors, inconsistencies, or improbabilities that cannot be automatically resolved. The consolidation engine may be configured to consolidate the first set of third party liability information and the second set of third party liability information after the data integrity engine has resolved or flagged any errors, inconsistencies, or improbabilities.

The system may comprise a third party liability engine. The third party liability engine may be configured to derive third party liability/settlement information for the identified patient from one or more information sources in one or more electronic repositories.

The system may comprise a delta engine. The delta engine may be configured to identify changes in third party liability guidelines. The delta engine may be configured to determine whether a particular change in third party liability guidelines applies to a particular patient.

The system may comprise an information query engine. The information query engine may be configured to search one or more information repositories electronically accessible by the system.

In some aspects, this disclosure relates to computer storage media embodying computer-executable instructions which, when executed by one or more processors, cause the one or more processes to perform a method for determining the existence of a third party payer/obligor. The method may comprise accessing a first set of third party liability information for an identified patient. The first set of third party liability information may be derived from a first electronic data source. The first set of third party liability information may include a first indication. The first indication may include one or more of an indication of an existing third party payer/obligor, an indication of an existing legal or judicial settlement, an indication of an existing promise or agreement by a third party to reimburse the healthcare provider for all or some part of the identified patient's healthcare costs, an indication of a first health condition of the identified patient, or an indication of a first healthcare service provided for the identified patient.

The method may comprise accessing a second set of third party liability information for the identified patient. The second set of third party liability information for the identified patient may be derived from a second source. The second set of third party liability information may include an indication. The indication of the second set of third party liability information may correspond to one or more of an indication of an existing third party payer/obligor, an indication of an existing legal or judicial settlement, an indication of an existing promise or agreement of a third party to reimburse the healthcare provider for all or some part of the identified patient's healthcare costs, an indication of a first health condition of the identified patient, or an indication of a first healthcare service provided for the identified patient.

The method may comprise characterizing one or more items from the first set of third party liability information for the identified patient to yield a first set of characterized items. The method may comprise characterizing one or more items from the second set of third party liability information for the identified patient to yield a second set of characterized items. The method may comprise determining whether any of the items in the first set of characterized items correspond to any of the items in the second set of characterized items. The method may comprise checking for errors, inconsistencies, or improbabilities in the values of any items from the first set of characterized items determined to correspond to an item in the second set of characterized items. The method may comprise resolving any errors, inconsistencies, or improbabilities, or flagging any errors, inconsistencies, or improbabilities that cannot be automatically resolved. The method may comprise consolidating the first and second sets of third party information to form a composite set of third party liability information for the identified patient.

The method may comprise processing the composite set of third party liability information for the identified patient to identify potential categories of third party liability. The method may comprise processing the composite set of third party liability information for the identified patient to identify potentially liable third party(s). The method may comprise selecting an item from the composite set of third party liability information, and assigning the item a weight according to a score for reliability of the information, and identifying potentially liable third party(s) based at least in part on the weighted item.

### BRIEF DESCRIPTION OF DRAWINGS

The disclosure makes reference to the attached drawing figures, in which:

FIG. 1 is a schematic illustration of an exemplary computing system environment useful in the practice of some aspects of the disclosure;

FIG. 2 is a schematic illustration of an exemplary method for consolidating third party liability information in accordance with aspects of the disclosure;

FIG. 3 is an illustration of an exemplary sub-process for characterizing and/or comparing data items in accordance with aspects of the disclosure;

FIG. 4 is a schematic illustration of an exemplary method for anomaly spotting in accordance with aspects of the disclosure;

FIG. 5 is a schematic illustration of an exemplary method for prompting a patient to correct information in accordance with aspects of the disclosure;

FIG. 6 is a schematic illustration of an exemplary method for assessing and/or improving reliability of third party payer/obligor identification in accordance with aspects of the disclosure;

FIG. 7 is a schematic illustration of an exemplary method for identifying and/or assessing third party liability corresponding to a patient in accordance with aspects of the disclosure;

FIG. 8 is a schematic illustration of an exemplary method for generating third party liability information corresponding to a patient in accordance with aspects of the disclosure;

FIG. 9 is a schematic illustration of an exemplary method for handling changes in third party payer/obligor guidelines in accordance with aspects of the disclosure;

FIG. 10 is a schematic illustration of an exemplary method for facilitating acquisition of patient consent, preferences, and/or information in accordance with aspects of the disclosure;

FIG. 11 is a schematic illustration of an exemplary method for facilitating monitoring of a patient's third party liability information in accordance with aspects of the disclosure;

FIG. 12 is a block diagram of an exemplary computing system in accordance with aspects of the disclosure;

FIG. 13 is a block diagram of an exemplary health information handling system in accordance with aspects of the disclosure; and

FIG. 14 is a block diagram of a health information handling system in accordance with aspects of the disclosure.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

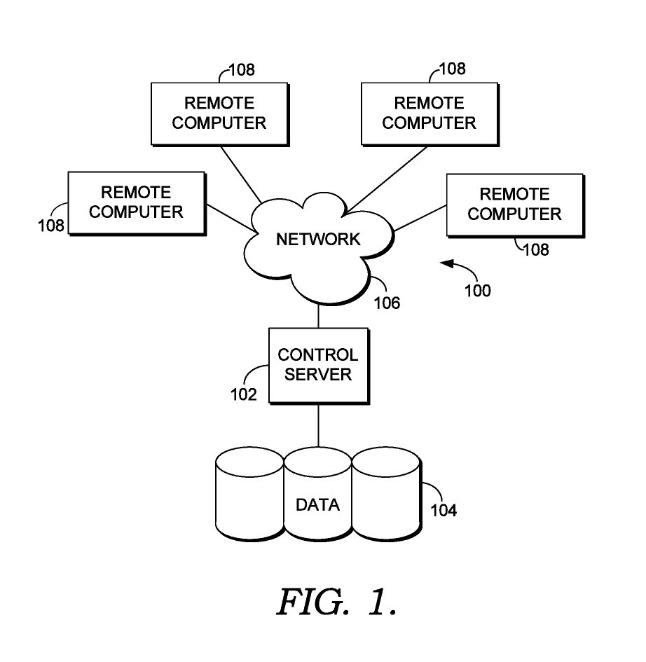

Referring to the drawings in general, and initially to FIG. 1 in particular, an exemplary computing system environment, for instance, a medical information computing system, on which embodiments of the present invention may be implemented is illustrated and designated generally as reference numeral 100. It will be understood and appreciated by those of ordinary skill in the art that the illustrated medical information computing system environment 100 is merely an example of one suitable computing environment and is not intended to suggest any limitation as to the scope of use or functionality of the invention. Neither should the medical information computing system environment 100 be interpreted as having any dependency or requirement relating to any single component/module or combination of components/modules illustrated therein.

Embodiments of the present invention may be operational with numerous other general purpose or special purpose computing system environments or configurations. Examples of well-known computing systems, environments, and/or configurations that may be suitable for use with embodiments of the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

Embodiments of the present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in local and/or remote computer storage media including, by way of example only, memory storage devices.

With continued reference to FIG. 1, the exemplary medical information computing system environment 100 includes a general purpose computing device in the form of a server 102. Components of the server 102 may include, without limitation, a processing unit, internal system memory, and a suitable system bus for coupling various system components, including database cluster 104, with the server 102. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The server 102 typically includes, or has access to, a variety of computer-readable media, for instance, database cluster 104. Computer-readable media can be any available media that may be accessed by server 102, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media implemented in any method or technology for storage of information, such as computer-readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 110. Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer-readable media. Computer storage media may exclude signals per se.

The computer storage media discussed above and illustrated in FIG. 1, including database cluster 104, provide storage of computer-readable instructions, data structures, program modules, and other data for the server 102.

The server 102 may operate in a computer network 106 using logical connections to one or more remote computers 108. Remote computers 108 may be located at a variety of locations in a medical or research environment, for example, but not limited to, clinical laboratories, hospitals and other inpatient settings, veterinary environments, ambulatory settings, medical billing and financial offices, hospital administration settings, home health care environments, and clinicians' offices. The remote computers 108 may also be physically located in non-traditional medical care environments so that the entire health care community may be capable of integration on the network 106, and/or in non-medical environments, such as insurance companies, attorneys' offices, or government agencies. The remote computers 108 may be personal computers, servers, routers, network PCs, peer devices, other common network nodes, or the like, and may include some or all of the elements described above in relation to the server 102. The devices can be personal digital assistants or other like devices.

Exemplary computer networks 106 may include, without limitation, local area networks (LANs) and/or wide area networks (WANs). Such networking environments are commonplace in offices, enterprise-wide computer networks, intranets, and the Internet. When utilized in a WAN networking environment, the server 102 may include a modem or other means for establishing communications over the WAN, such as the Internet. In a networked environment, program modules or portions thereof may be stored in the server 102, in the database cluster 104, or on any of the remote computers 108. For example, and not by way of limitation, various application programs may reside on the memory associated with any one or more of the remote computers 108. It will be appreciated by those of ordinary skill in the art that the network connections shown are exemplary and other means of establishing a communications link between the computers (e.g., server 102 and remote computers 108) may be utilized.

In operation, a user may enter commands and information into the server 102 or convey the commands and information to the server 102 via one or more of the remote computers 108 through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote healthcare device to the server 102. In addition to a monitor, the server 102 and/or remote computers 108 may include other peripheral output devices, such as speakers and a printer.

Although many other internal components of the server 102 and the remote computers 108 are not shown, those of ordinary skill in the art will appreciate that such components and their interconnection are well known. Accordingly, additional details concerning the internal construction of the server 102 and the remote computers 108 are not further disclosed herein.

Although methods and systems of embodiments of the present invention are described as being implemented in a WINDOWS operating system, operating in conjunction with an Internet-based system, one of ordinary skill in the art will recognize that the described methods and systems can be implemented in any system supporting the automated configuration, implementation and/or maintenance of a healthcare information system. As contemplated by the language above, the methods and systems of embodiments of the present invention may also be implemented on a stand-alone desktop, personal computer, or any other computing device used in a healthcare environment or any of a number of other locations.

As used herein, “billing system” refers to a computer application utilized by a computer or other electronic device for electronic submission of, or electronic receipt of, healthcare claims to Federal, State or commercial health insurers or other third parties (i.e., not the healthcare provider or the patient) who may be responsible for part or all of a patient's healthcare expenses.

As used herein, “common working file” refers to a tool used by the Centers for Medicare & Medicaid Services (CMS) to maintain national medical records for individual beneficiaries enrolled in any of the programs provided by CMS. The system is used to determine the eligibility of patients and to monitor the appropriate usage of medical benefits by such patients. The common working file retains a record for each patient, which is automatically created by the government upon the patient enrolling in one or more programs offered by CMS. The record documents claim history, benefits and eligibility for the patient in order to expedite the prepayment process. In addition, the record contains important personal health information for the patient, such as date of birth and/or death, information about secondary or additional insurance coverage and additional health and liability information. Preferably the records are in digital/electronic form.

As used herein, “credentials” refers to information, objects, or characteristics a user must know or possess in order to gain entry to a restricted access system. Non-limiting examples, include, but are not limited to: security tokens, usernames, passwords, fingerprints, retinal scans, other biometric identifiers and other methods of authentication.

As used herein, “consolidation engine” refers to a computerized or electronic system specifically programmed for consolidating sets of confidential health information and/or payer/third party liability information for a particular patient. The consolidation engine may utilize one or more processors, together with one or more network interfaces, to push and/or pull third party liability and/or confidential health information from one or more of the healthcare provider's, the healthcare payer(s), the personal data sources, and/or the other data sources, through the network. In addition to, or in lieu of, transferring information over the network, third party liability or health information could be pre-loaded and/or directly transferred to the health information handling system (e.g., via a storage medium). The consolidated data may be retained in one or more repositories or databases.

As used herein, a “data integrity engine” refers to a computerized or electronic system specifically programmed for checking third party liability and/or health information to ensure the quality or accuracy of the data underlying the identification of a third party payer/obligor for a particular patient. The data integrity engine may assess each piece of information underlying the identification of a potentially liable third party and may assign a weight to the information according to a score. The data integrity engine may utilize one or more network interfaces to convey the results of the third party liability scoring to a user.

The data integrity engine may examine items of information and assign scores according to how important such informative is to attributing third party liability generally. The data integrity engine may adjust scoring of information in view of specific third party liability information for a given patient. The data integrity engine may examine items of information in view of specific information regarding a third party payer/obligator upfront, thereby rendering subsequent readjustment unnecessary. Based on the scoring, possible follow-up questions and/or prompting for further information and/or clarifying information may be identified, generated, and/or provided.

As used herein, a “database” refers to a computer database that electronically stores records and files, which may be created, modified and/or accessed by those with the proper credentials.

As used herein, a “delta engine” refers to a computerized or electronic system specifically programmed to recognize changes in third party liability guidelines (e.g., those implemented by law/regulation, etc.). The delta engine may process the changes to identify exactly what has changed, the scope of the changes, and potential ramifications. As a non-limiting example, it may be determined that limitations on liability for premises liability have changed, and the delta engine may identify patients with outstanding claims which may be affected by that change.

The identification of affected patients may be based on the determinations that the changes affect certain third party obligations, liability, settlements, certain types of patients, certain payers, etc., and applying those determinations to the patient. The delta engine may notify providers and/or patients of those changes.

As used herein, “factor accounting” refers to the use of factors such as, but not limited to, age, geography, address, and/or other factors of the composite set to identify records through cross-comparison of these factors.

As used herein, “health information handling system” refers to any electronic device or set of electronic devices configured to compute, process, store, display, handle, and/or use any form of digital information and/or digital data suitable for the embodiments described herein. The electronic device(s) can be preferably used by the Healthcare Provider and can be located at the location of the Healthcare Provider or the location where the Healthcare provider houses their information technology equipment or it can be in electrical communication with such equipment but remotely located therefrom.

As non-limiting examples, the health information handling system could include a single computer server located at the healthcare provider or elsewhere, or multiple computing devices which may be implemented in or with a distributed computing and/or clouding computing environment with a plurality of servers and cloud-implemented resources. Thus, a health information handling system may include one or more servers. The health information handling system may include one or more processing resources communicatively coupled to one or more storage media, one or more electronic databases, random access memory (RAM), read-only memory (ROM), flash memory, and/or other types of memory. The health information system may include any one or combination of various input and output devices (I/O) devices, network ports, and display devices.

As used herein, “healthcare payer” refers to any person, entity, organization, institution, etc., that provides for payment related to healthcare, healthcare services, and/or claims for healthcare services, and/or that administers insurance and/or benefits. In some aspects, the healthcare payer may be a source of health information and/or payer benefit information and/or third party liability information relating to patients. By way of example without limitation, Medicare, a government entity, as a healthcare payer, may be a user that receives information whether through the network or otherwise, may provide information to the health information handling system and third party liability, and/or may have access to records, databases, and/or other repositories containing information about third party liability. Medicare's information repositories may be electronically linked to the health information handing system such that the repositories may correspond to the healthcare payer in some embodiments.

As used herein, “healthcare provider” refers to a hospital, company, medical service provider, facility, etc., who may provide healthcare services to patients.

As used herein, “information query engine” refers to a computerized or electronic system or method which may be configured to electronically search one or more information repositories. These searches may be initiated by a user or in response to information received over the network from a user. Responsive to the query, the information query engine may search, retrieve, modify, and/or cause transfer of particular information from one or more information repositories.

As used herein, “interface” refers to any suitable electronic input/output module or other electronic system/device operable to serve as an interface between the personal data sources and any other data sources and the network. The interface(s) may facilitate communication over the network using any suitable transmission protocol and/or standard.

As used herein, “Medicaid” refers to a national social insurance program, administered by the U.S. federal government, which provides health insurance for low-income persons of any age. It will be appreciated that in most instances, a reference to Medicaid could refer to any other government-sponsored and/or government-operated healthcare or insurance program.

As used herein, “Medicare” refers to a national social insurance program, administered by the U.S. federal government, and currently using about 30 private insurance companies across the United States. Medicare provides health insurance for Americans aged 65 and older who have worked and paid into the system, as well as some younger Americans with disabilities. It will be appreciated that in most instances, a reference to Medicare could refer to any other government-sponsored and/or government-operated healthcare or insurance program.

As used herein, “memory” or “memories” refer to physical or virtual devices and/or drives used to store programs, data, information, etc., on a temporary and/or permanent basis for use by a computer or other electronic device.

As used herein, “network” refers to any suitable means to facilitate data transfer in the system. Non-limiting examples of network types include one or more of the following: the Internet, a wide area network (WAN), a local area network (LAN), a wireless local area network (WLAN), an intranet, and a metropolitan area network (MAN). Devices may connect to the network using wired connections, wireless connections, or a combination thereof. Exemplary network connections include connectivity via a cellular network, such as 5G, 4G, 3G, GSM, etc., WiFi, Bluetooth®, Near Field Communication (NFC), satellite communications, or any other wireless network, Ethernet, Universal Serial Bus (USB), Firewire®, serial port, parallel port, remote desktop gateway, and/or any other appropriate architecture or system that facilitates the communication of signals, data, and or messages that is currently existing or developed in the future. The network may transmit data using any suitable wireless or wired communication protocol currently existing or developed in the future, including, without limitation, Transmission Control Protocol (TCP), User Datagram Protocol (UDP), or Internet Protocol (IP). The network and its various components may be implemented using hardware, software, and communications media such as, but not limited to, wires, optical fibers, microwaves, radio waves, and other electromagnetic and/or optical carriers, and any combination of the foregoing.

As used herein, “patient” refers to a person under medical care or treatment from a healthcare provider.

As used herein, “portal” refers to a website, webpage, or other similar electronic/digital medium through which read/write access to data may be granted.

As used herein, “processor” refers to electronic circuitry within a computer, tablet, smart phone, other similar electronic device, etc., that carries out the instructions of a software program by performing the basic arithmetic, logical, control and input/output (I/O) operations specified by the instructions.

As used herein, “reimbursement rate” refers to the rate at which a healthcare payer (e.g., Medicare, etc.) pays for healthcare services.

As used herein, “repository” refers to a database, electronic, physical, or otherwise where information, data, files, etc., may be stored and accessed by those with the proper credentials.

As used herein, “settlement” refers to a resolution between disputing parties, which may be reached before or after a legal action or arbitration proceeding is initiated regarding the dispute. As a non-limiting example, a settlement could include an agreement for one party to pay the healthcare costs of a patient.

As used herein, “third party liability engine” refers to a computerized or electronic system or method which may provide a provider with third party liability/settlement information corresponding to a patient(s). The third party liability engine may derive third party liability/settlement information for the identified patient(s), such as, but not limited to, by pulling and/or pushing third party liability information from one or more of the following: healthcare providers, healthcare payers, personal data sources, and/or any other data sources—or by processing preloaded and/or otherwise directly transferred third party liability/settlement information. The third party liability engine may access third party liability information, such as, but not limited to, settlement information stored in one or more third party liability repositories.

The third party liability engine may access one or more sources to identify a potentially liable third party such as, but not limited to, by crawling through available information repositories. The third party liability engine may identify a specific third party or third parties liable for payment corresponding to the identified patient. The third party liability engine may correlate confidential health information and/or liability information to a set of criteria in view of the patient's healthcare costs.

As used herein, “third party payer/obligor” refers to a person, company, organization, entity, facility, etc., other than the patient or healthcare provider who may be liable or responsible for paying all or part of a patient's healthcare costs. Non-limiting examples include the insurance company of an individual who caused an injury to the patient or an individual personally responsible. The insurer or the individual personally may then be liable or responsible for all or part of the patient's healthcare costs.

As mentioned briefly above, matching liability claims to healthcare claims can be a time-consuming and challenging process. Liability claims, such as legal or liability insurance claims, may be processed through different providers (e.g., courts, attorneys, different insurance companies or different departments of a particular company) using different information repositories and information handling systems than claims related to the costs of healthcare for an individual, e.g., as may be submitted for payment by a healthcare provider. Confidentiality requirements in both the legal and healthcare industries may complicate data sharing between unrelated service providers for a particular patient. A patient may not have any information regarding potential third-party liability at the time of medical treatment, e.g., if a patient needs immediate medical assistance with an injury, and investigates a legal or liability insurance claim after the initial treatment or during or after a course of treatment. This information may become available from various information sources between the time(s) of treatment and the preparation of a claim for payment of healthcare costs.

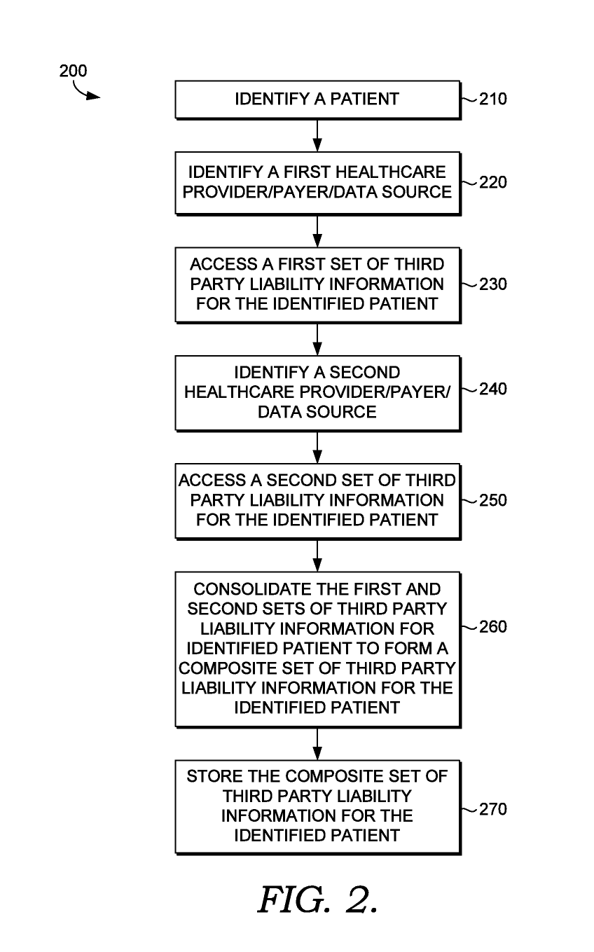

FIG. 2 illustrates a non-limiting example for a method 200 of consolidating third party liability information. The relevant information may be under regulatory control from healthcare entities and patients. Teachings of the present disclosure may be implemented in a variety of configurations that may correspond to the systems disclosed herein. As such, certain steps of the method and other methods disclosed herein, may be omitted, and the order of the steps may be shuffled in any suitable manner and may depend on the implementation chosen. Moreover, while the flowing of the preferred steps for the method of FIG. 2, and those of other methods disclosed herein, may be separated for the sake of description, it should be understood that certain steps may be performed simultaneously or substantially simultaneously.

The method 200 may begin when a particular patient is identified by the healthcare provider or health information handling system, shown as step 210. At step 220, a first data source is identified. The data source may correspond to one or more of the following: a healthcare provider, a healthcare payer, a personal data source, a third party payer/obligor, a common working file, a repository of one or more of those entities, and/or other information corresponding to healthcare services provided to the patient. Data may be actively gathered and/or pulled from one or more data sources, such as, but not limited to, by accessing a third party repository. Data could be gathered by electronically “crawling” the various repositories in some embodiments.

At step 230, a first set of third party liability information for the identified patient may be accessed. The information may have come from the first data source and may indicate the liability of a third party for all or any portion of a patient's medical expenses owed to a healthcare provider for the particular patient. The information may have been retained in a memory and/or repository before step 230; alternatively, the information may be retained in a memory or repository simultaneously or consequent to step 220. The repository can be of third party liability information or other set of information/data which may contain information about whether there is a third party payer/obligor. The repositories can provide third party liability information from various data sources and allow such information to be collected for use by the disclosed system and method. As non-limiting examples, the first data source may include one or more diagnosis codes, such as ICD9 codes; information about the cost-to-date of the patient's healthcare for a particular diagnosis, injury or complaint; requests for information or copies of medical records to be sent to non-medical service providers; court records; and/or secondary insurance information.

The data from the first data source could directly indicate that a third party is liable, but more likely provides clues that a third party might be liable. For example, certain kinds or combinations of injuries, such as chemical burns to the face or a broken arm and a broken leg, may be more likely to result from an accident involving third party liability than some other kinds of conditions, such as a sprained ankle (in isolation) or a minor cut on the hand (in isolation). As another example, prior third party payments for healthcare services related to a diagnosis code for a particular injury make it more likely that the same third party may be liable for the cost of additional healthcare services related to the same diagnostic code. As another example, a request to send medical records to an attorney or a non-healthcare insurance department or company (such as an automobile insurance company or a commercial insurer) may be indicative of third-party liability. As yet another example, a lawsuit filed in the patient's name and county of residence may make it more likely that a third party is liable for some or all of the cost of the patient's healthcare.

If no indication of possible third party liability is located in the first data source, the process may end for this patient and/or the current medical bill. Alternately or additionally, the process could be re-initiated for this patient and/or the current medical bill at a later time, when third party liability information might have been added or updated in one or more data sources. For example, a medical bill might be held for further analysis before being released to the patient, patient's health insurer, or other payer. In the first or later iterations of the process, a second and/or subsequent data sources may be analyzed for indications of possible third party liability even if no indication of possible third party liability is identified in the first data source.

At step 240, a second data source is identified. Again, the second data source may correspond to one or more of the following: a healthcare provider, a healthcare payer, a personal data source, a third party payer/obligor, a repository of one or more of those entities, and/or other information corresponding healthcare services provided to the patient. As a non-limiting example, one type of repository of potential relevant information can be the Medicare Common Working File.

At step 250, a second set of third party liability information for the identified patient may be accessed. The second set of third party liability information may be different from the first set of third party liability information. Different sets of information could be derived from the same data source, for example, by way of one or more updates to information previously provided by a particular data source for a particular patient. The second data source may contain similar kinds of information as the first data source. For example, the first and second data source may both contain healthcare service records. The second data source may contain different kinds of information as the first data source, or may contain a mix of similar and dissimilar kinds of information relative to the first data source. The sets could include, as a non-limiting example, indications of third party liability to a particular patient and/or for a health care service(s) or product(s) that has been provided to a patient. Different sets of information could be derived from different data sources, even if the different data sources contain similar kinds of information.

At step 260, the first and second sets of third party liability information may be consolidated to form a composite set of third party liability information for the identified patient. The consolidation may include organizing, categorizing, qualifying, and/or comparing the sets of information; detecting, identifying, and/or handling errors/discrepancies; and/or otherwise processing the sets of confidential health information for the identified patient. The consolidation is not merely the identification of previously classified or tagged information, but may use context-based searching to identify, collect, and categorize potentially relevant information that was not previously identified as relevant for this purpose.

At step 270, a composite set of third party liability information for the identified patient may be stored. For example, the composite set of information may be retained in memory and/or repository of or associated with the health information handling system.

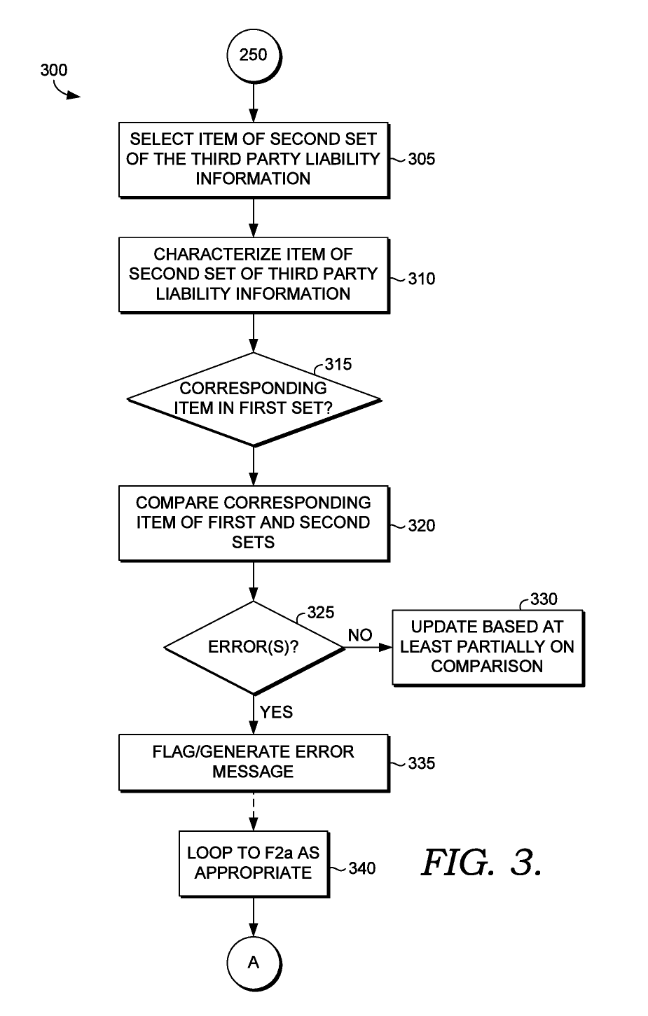

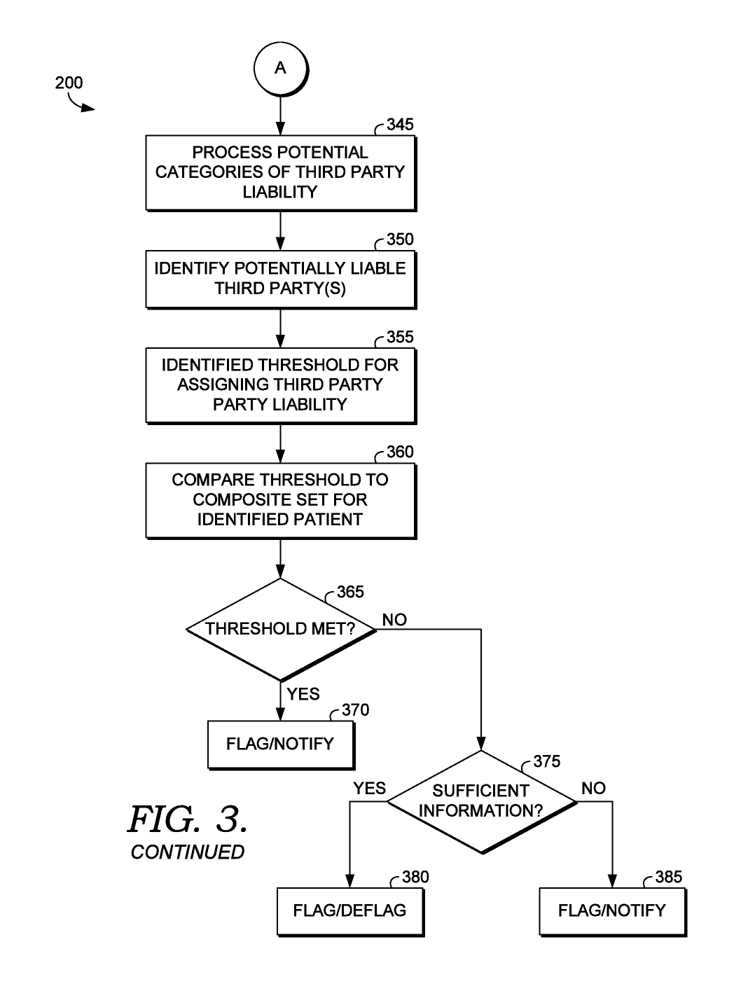

FIG. 3 is an illustration of a non-limiting example of a sub-process 300 or subroutine corresponding to step 260 of the method illustrated in FIG. 2, in accordance with some embodiments of the present disclosure. The sub-process 300 shown in FIG. 3 may be performed by the health information handling system.

At step 305, an item from the second set of third party liability information can be selected. As step 310, the selected item from the second set of third party liability information may be characterized. As a non-limiting example, an item of third party liability information may be characterized/identified as one or more of the following: belonging to a particular category of information (e.g., type of third party payer/obligor), and/or belonging to a particular subcategory of information (e.g., name of insurer). Some embodiments may characterize the item of information further, for example as coming from a particular source and being qualified accordingly (e.g., information may be characterized as coming from a more or less reliable source), being more or less important than other items of information, and/or according to how recent the information is. These characterizations may be performed by the electronic system, without human input or assistance. For example, the electronic system may use context-based searching, pattern recognition in the data, and/or the application of rules and logic to characterize the information, which may have previously been unclassified, or using classifications intended for a different purpose which are not relevant in this context. That is, the system may itself characterize the information for the first time, or may change the characterization initially associated with the information.

At step 315, the system may determine whether there is an item of information in the first set of third party liability information for the identified patient that corresponds to the selected and characterized item from the second set of third party liability information for the identified patient. The determination may be made based in part of the characterizations of the items. In the case of no corresponding items in the second set being identified, the item could be new information in a category for which no information had been previously received (e.g. the new information could indicate a settlement agreement was reached, whereas that could have been previously unknown to the health information handling system); and the flow may proceed to step 325.

In the case of a corresponding item in the second set being identified, the items from the first and second set may be compared by the health information handling system, at step 320. These comparisons may serve one or more purposes, or different comparisons may be made for different purposes. As one example, patient identifiers may be compared to confirm that the records being analyzed are for the same person. In some instances, this comparison may be a straightforward comparison of, for example, the patient's social security number in each data source. In other instances, it may be necessary to compare the information indirectly, e.g., to match a medical record number or insurance identification number to a patient's name and/or address in a different record. As another example, information may be compared to determine whether the information is related, for example, comparing a diagnostic code for a recent, unbilled healthcare service to a diagnostic code for a healthcare service recently paid by a third party. If the diagnostic codes are the same, the likelihood that the third party is also responsible for the new healthcare costs increases.

The system may seek additional information, e.g., to confirm the maximum payments under an insurance policy or judgment. If the diagnostic codes are different, the system may seek information about whether or not the diagnoses are related. The system may search or crawl certain data sources based on the information available. For example, if a potentially liable third party has been identified from the first data source, the system may search as the second or subsequent data source payer or other data sources that are likely to disclose the maximum payments the third party is liable for. In some instances, if the system cannot find information that is logically necessary or useful to determine third party liability for a healthcare expense, the system may flag the file for review by a human user and/or send an electronic inquiry about the file. For example, the system may prompt a healthcare provider to answer a question about whether a head injury and a neck injury are related by cause, or may send an inquiry via a payer interface regarding an insurance policy's minimum and/or maximum payment thresholds.

At step 325, the system may determine if any errors are detected in the form and content of the item(s) of information. As a non-limiting example, data stored in a repository can be presented in a non-standardized format. A non-standardized format may be a format that is not readable or understandable by the system, such as pictures, tiff images, non-ocr pdf files, x-rays, or other graphic-oriented or non-machine readable formats. Data may also be treated as non-standardized if the system cannot map or compare the data for some reason, such as the data being in a nomenclature new to the system. In some instances, the system may be able to generally characterize the data, e.g., as radiology images or a kind of patient or payment data typically associated with a particular file type, but may be unable to “read” the data in manner that would allow the system to compare the data to data from other files or sources.

In the case where the system encounters data in a non-standardized, unrecognized, or unreadable format, the system administrator may be alerted that the data cannot be compared electronically, and the user may manually amend or enter the necessary information to perform the comparison shown in step 320. In the case of no errors, the composite set of information may be formed/updated at step 330, based at least partially on the comparison performed in step 320. As a non-limiting example, the system may present the information in the form of a standardized report presenting third party liability information in a method compatible with a healthcare provider's billing system. For example, the standardized report may be prepared as a csv, xml, txt or xls file. The data provided and the format or layout of the data may vary independently of the file type (e.g., csv), for example, based on user preferences or changing industry practices. If any errors are detected, the errors are flagged at step 335. In some aspects, an error message may be generated and can be sent to a system administrator.

If there are additional items of information available, the process flow may loop at step 340 back to step 305 for processing of those other items of information. After appropriate iteration with looping back through the previous steps, the flow may move forward to processing potential third party liability categories at step 345. Third party liability may be categorized according to a coding method, where a given third party is assigned a letter grade, such as A, B, C, etc. Though not limiting, the grade can be assigned based on the extent of the third party's liability to pay the patient's medical expenses. The system can attribute the grade though such is not considered limiting and the grade can also be manually made or automatically made. The third party liability can be admitted by the particular third party or the particular third party has been found liable or responsible for payment by a medical or judicial determination. Grade levels A and B, for example, may entail relatively mechanical determinations, such as when a third party has admitted liability in a particular case. The third party liability categories may be processed in view of the composite set of third party liability information for the identified patient. In a non-limiting embodiment, the system can review the Medicare Common Working File (CWF) or other documents to identify whether there is a third party liable for payment of some or all of the patient's medical expenses. Once a third party is identified at step 350, step 345 then examines to what extent that party is liable and assigns them a grade accordingly. In this regard, steps 345 and 350 may be performed concurrently or iteratively, or step 350 may be performed before step 345. The third party liability categories may be filtered with criteria such as extent of liability, extent of coverage, etc. to identify potential third parties who may be liable for a patient's health care coverage. As a non-limiting example, it may be determined that any third party liable for the entirety of a patient's healthcare costs would receive a grade of A, third parties liable for 80%-99% of a patient's healthcare costs would receive a grade of B, third parties liable for 60%-79% of a patient's healthcare costs would receive a grade of C, and any liability 59% and below might receive a D or F. Other ranges, percentages, etc. can also be used and are considered within the scope of the disclosure.

At step 355, one or more thresholds for third party liability may be identified. One non-limiting example can be where a particular third party may be liable for a patient's healthcare coverage if the patient's healthcare costs exceed a certain dollar amount.

At step 360, the threshold may be compared to the composite set of health information and third party liability information for the patient by the health information handling system. As a non-limiting example, it may be determined that the third party is liable for all or a portion of an identified patient's healthcare costs, where a third party's healthcare costs have exceeded the threshold identified in step 355.

At step 365, the system determines whether the threshold for liability is met. If the threshold is met, the third party may be flagged for payment/reimbursement and/or the patient and/or healthcare provider/payer may be notified at step 370. If the threshold is not met, it may be determined whether sufficient information is available in the health information handling system to determine with certainty that the threshold has not been met at step 375. If sufficient information is available, the third party may be flagged or de-flagged at step 380. For example, a third party may be flagged if there is sufficient information to indicate that the third party would be liable if the patient's healthcare costs ultimately meet or exceed the threshold amount. In other instances, the third party may be de-flagged as not be liable for one or more reasons. As a non-limiting example, the patient may be fully at fault for the injuries sustained, and a third party insurer would therefore not be obligated to cover the patient's healthcare costs.

If sufficient information is not available, the patient and/or healthcare provider/payer may be notified at step 385. Thus, where there are gaps in the information, the gap instances are noted in order to prompt for getting that information.

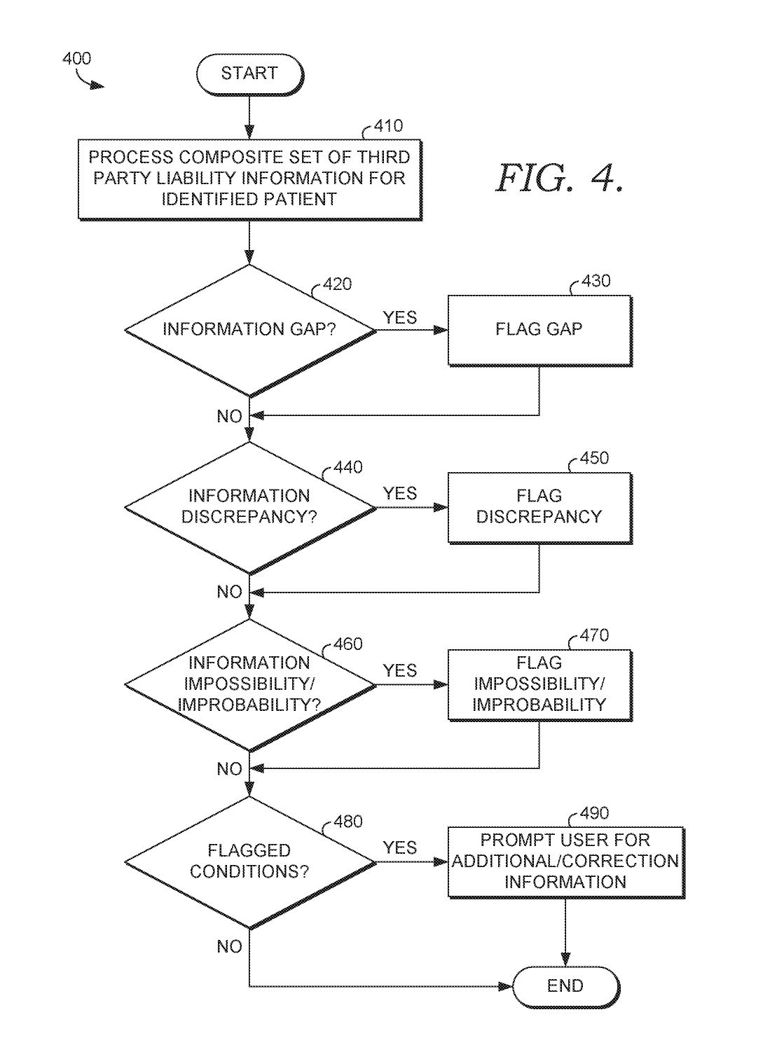

FIG. 4 illustrates a non-limiting method 400 for anomaly spotting by identifying gaps in information, conflicting information, impossible/improbable information, and/or similar records that may be related, in accordance with some embodiments in the present disclosure.

Some aspects of this method may be performed by the health information handling system and the data integrity engine. Certain data issues can be solved by comparing datasets that do not exactly match. As one non-limiting example, there may be discrepancies in the legal name of a third party, or the amount to which that party is liable. The system may include an algorithm that takes into account age, geography, address, and or/other factors that can be used to identify similar records that might be linked together.

Method 400 may start with step 410, where a composite set of third party liability information for an identified patient may be processed. As a non-limiting example, the health information handling system may access third party liability information which may have been stored in a repository. The repository may be on-site, off-site and/or located remotely of the health information handling system and/or data integrity engine. The system and/or engine may access more than one repository.

At step 420, the system may determine whether one or more information gaps exist in the composite set. As a non-limiting example, the health information handling system may have a standardized form for reporting third party liability information including the third party's name, address, the scope of their liability, and any additional information relevant to obtaining reimbursement from that third party. As one non-limiting example, a third party payer/obligor's billing address may be missing for the identified patient. Different and/or additional information could be used in a standardized form and/or alternate reporting template. Any gaps in identified in step 420 may be electronically flagged by the system in step 430. The user may be notified that there is information missing necessary to identify and subsequently bill a potentially liable third party.

At step 440, it may be determined whether one or more discrepancies exist in the composite set. As one non-limiting example, there may be naming and identification issues, such as multiple spellings of the name of the identified third party payer/obligor. This determination can be made manually, or electronically by the system. Any discrepancies identified in step 440 may be electronically flagged by the system in step 450. The user may be notified there is information missing necessary to identify and subsequently bill a potentially liable third party.

At step 460, it may be determined whether one or more impossibilities/improbabilities exist in the composite set. As one non-limiting example, there may be two or more third parties liable for the entirety of the identified patient's medical expenses. If not an impossibility in real life, this makes it impossible for the system to determine which of the two or more third parties should be billed for the medical expenses. Any impossibilities/improbabilities identified in step 460 may be electronically flagged by the system in step 470. The user may be notified there is a discrepancy in the third party liability information and prompted to make the necessary correction.

At step 480, the system considers any flagged conditions. If there were no flagged conditions per steps 430, 450, and/or 470, the process flow may end. If there is a flagged condition per steps 430, 450, and/or 470, a user may be promoted for additional/correcting information at step 490.

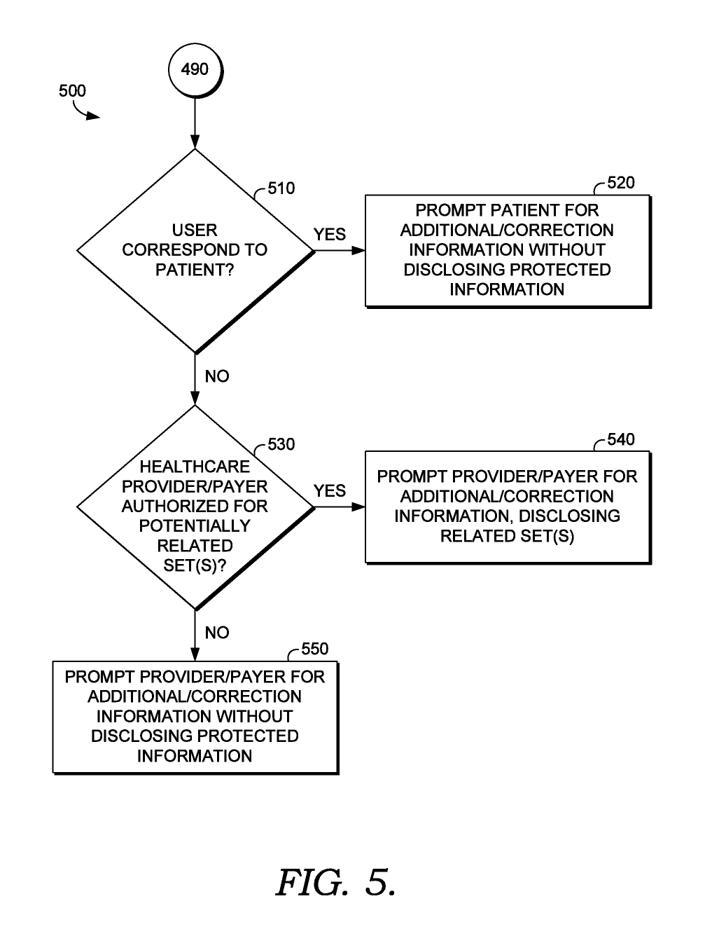

FIG. 5 illustrates a non-limiting example of how a patient might be prompted to correct information, in the form of a sub-process 500 or subroutine corresponding to step 490 of FIG. 4, in accordance with some embodiments of the present disclosure. Prompted by flagging, the provider, and under some circumstances, the patient, can correct errors and/or add additional information. This may be done in a way that obscures protected information, for example, information protected by the Health Insurance Portability and Accountability Act of 1996 (HIPAA) or other relevant law or regulation. Treatment of such information might be different between the patient and provider, according to some embodiments.

In step 510, it may be determined whether a user accessing confidential health and/or third party liability information corresponds to the identified patient or legal representative thereof. If the user is the identified patient or legal representative thereof, the user may be promoted for additional/correction information in step 520. In some embodiments, the user may be prompted for additional or corrective information without displaying and/or requiring the user to disclose protected or confidential information. If the user is the patient or patient's legal representative, the system may solicit and/or display protected or confidential information as relevant to user actions and/or prompts. If the user does not correspond to the identified patient, it may be determined whether the user is a healthcare provider or payer that is authorized to access the composite set of the identified and the potentially related information set(s) at step 530.

If the user is a healthcare provider or payer that is authorized to access the composite set of the identified and the potentially related information set(s), the healthcare provider/payer may be prompted for additional/correction information, and the potentially related information set(s) may be disclosed to/provided for access by the healthcare provider/payer, at step 540.

In the case of a healthcare provider/payer or other user not being authorized to access the composite set of the identified and the potentially related set(s), the user may be prompted for additional/correction information, without disclosing protected or confidential information, at step 550.

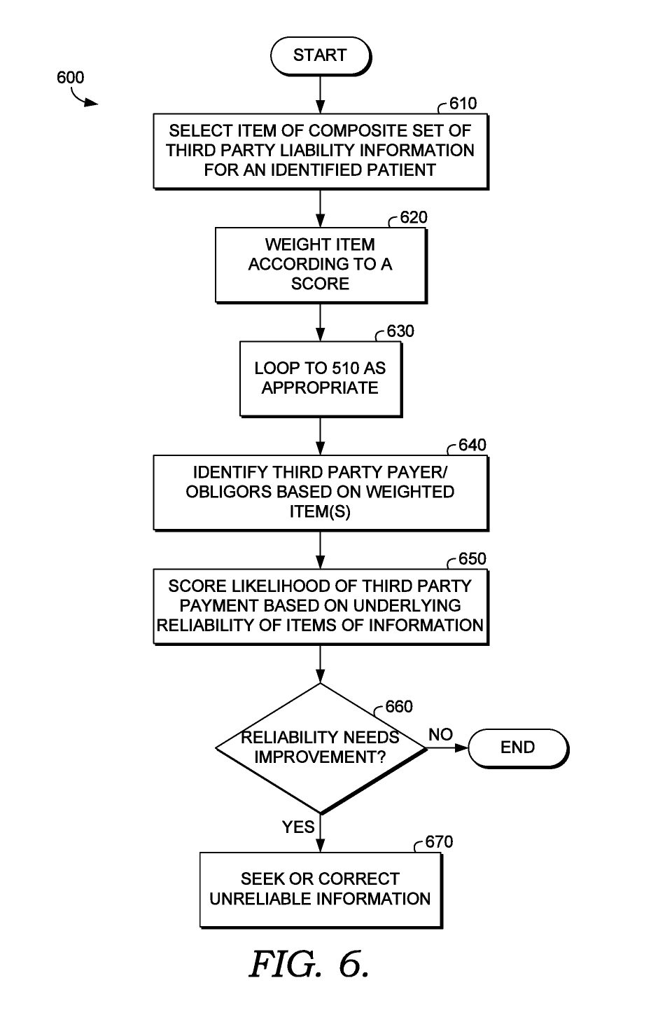

FIG. 6 illustrates a non-limiting method 600 for assessing/improving reliability of identifying third party payer/obligors in accordance with some embodiments of the present disclosure. Alternatively or additionally to third party payer/obligor identifications, some embodiments may also identify settlements or other legal or judicial agreements (e.g., third party payer/obligors who might be liable for causing the injury, as well as third party payer/obligors bound by contractual or other obligations to the patient). Some or all aspects of identifying third party payer/obligors may be performed by the health information handling system and the data integrity engine. Third party liability information may be affected by poor underlying data in the medical record or other related files. In some aspects, each piece of underlying third party liability information may be weighted according to a score. Documents, files, records, etc. (“records”) having missing information, for example, could have a lower score than records not missing information; and records missing information can be scored even lower depending on the type of information missing. The more important the missing information is to assessing and/or ascribing third party, the lower the score for the record may drop. Third party liability information may be scored based upon the underlying reliability to avoid redundant billing, or billing payers with lower reimbursement rates. Third party liability can be more reliably assessed with the provider asking possible follow-up questions and/or prompting for an account to link to where at least some of the missing third party liability information can be found. Therefore, the accuracy of the third party liability information can be assessed and corrected if needed.

Method 600 may begin with step 610, in which an item of the composite set of third party liability information for an identified patient may be selected. In step 620, the selected item may be weighted according to a score. Missing information, for example, could have a lower score than non-missing information; and the missing information could be scored even lower, the more important the information is to assessing and/or ascribing third party liability. For example, records with missing information could have a lower score than complete records. Records missing more critical information can receive a lower score than records missing non-critical information. Third party liability information may be scored based upon the underlying reliability to avoid redundant billing, or billing payers with lower reimbursement rates. In alternative or in combination, information may be weighted according to the source. For example, in some instances, information gathered from a third party insurer may be weighted higher or lower relative to information gathered from a patient.

At step 630, the process flow may loop back to step 610 for processing of other items of information. After appropriate iteration with looping back through the previous steps, the process flow may transition to step 640.

At step 640, third party payer/obligors may be identified and/or ascribed liability based on the weighted items. For one example, the name of a third party may be identified within a record examined by the health information handling system or the data integrity engine. There may be further information contained within a record as to the identity of the third party, the scope of their liability, the rate at which the healthcare provider could be reimbursed from that party and other similar information.

At step 650, the likelihood of payment from the identified third party may be scored based on an underlying reliability of the items of information. The scoring of the information items may be adjusted in view of the source of information regarding an identified third party's liability. For example, whereas an item of information may be accorded a certain score generally, the item may be important in view of a specific admission by that third party and thus, may be scored accordingly.

At step 660, it may be determined whether the reliability of the information underpinning the identification and/or assessment of liability on a third party needs improvement. This determination may be based at least in part on the weighting described in step 620, and whether the weight or score assigned to the information exceeds a threshold level. The threshold itself may be determined by the billing entity, based on the billing entity's tolerance for uncertainty. If no improvement is required, the process may end. If so, a healthcare provider may be prompted to provide information needed to improve the reliability of the identification and/or assessment at step 670. In an alternative or in combination, one or more data source(s) may be accessed to obtain information to improve the reliability of the identification and/or assessment of third party liability.

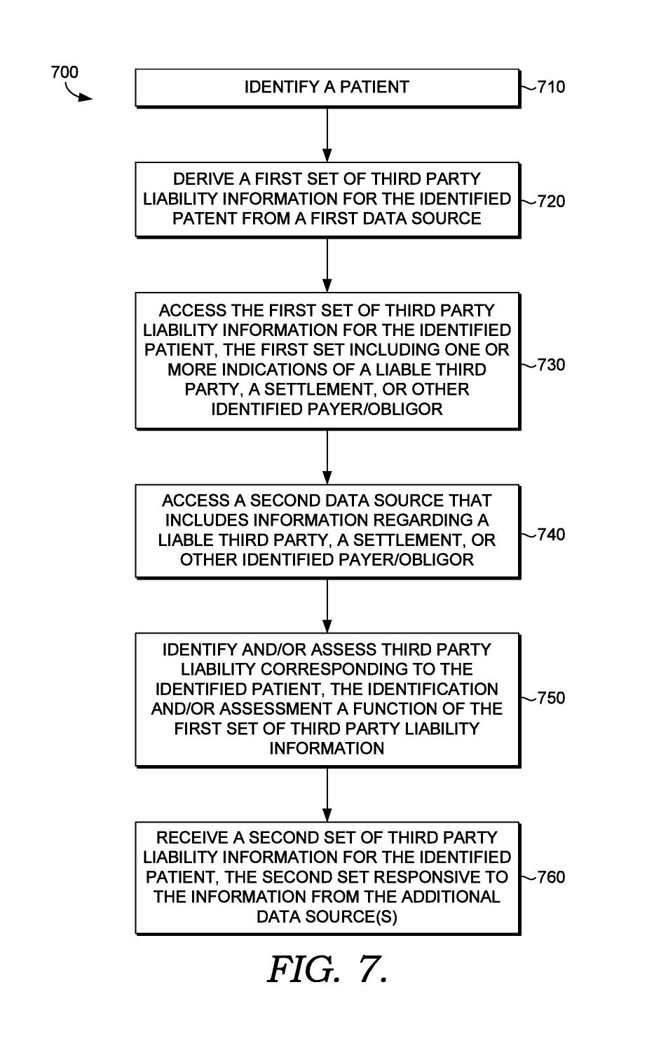

FIG. 7 illustrates a method 700 for identifying/assessing third party liability corresponding to a patient, in accordance with some embodiments of the present disclosure. Alternatively or additionally to third party payer/obligor identifications, in some aspects, the system may operate with respect to settlement or other legal or judicial agreements that may also create payment liability by a third party to a patient or other related person.

The third party liability identification/assessment process may be responsive to a request made by the healthcare provider. In some embodiments, the third party liability identification/assessment process may be initiated by another user such as the patient or an agent or contracted service provider of the healthcare provider. First, a patient is identified is identified at step 710. The third party liability/identification assessment process may be automatically initiated, for example, based on a set schedule or on an event such as a new information update. New information may come from various sources, including, but not limited to, the Medicare common working file, a judicial decision, a settlement, etc.

At step 720, a first set of third party liability information may be derived from a first data source. By way of non-limiting example, a first set of third party liability data for the identified patient may be pulled and/or pushed from Medicare's Common Working File (CWF), a database maintained by Medicare and/or Medicaid, the healthcare providers/payers data sources, or may be pre-loaded by Medicare and/or Medicaid, the healthcare providers/payers data sources or other party and/or otherwise directly transferred by any of the aforementioned parties. This information may be stored in one or more database(s)/memory(ies), or repository(ies) associated with the Health Information Handling System.

At step 730, the first set of third party liability information for the identified patient may be accessed by the Health Information Handling System. As a non-limiting example, the first set of third party liability information may be retrieved from one or more third party liability information repositories such as database(s)/memory(ies) associated with the Health Information Handling System, the third parties themselves, or another provider of information such as Medicare's Common Working File (CWF) or a database maintained by Medicare and/or Medicaid. The first set may include any known third party liability information for the identified patient. The first set may include, without limitation, one or more indications of the existence of a third party payer/obligor, a settlement agreement, an admission of liability, and/or any other pertinent information regarding a potential third party payer/obligor.

At step 740, a second data source may be accessed by the Health Information Handling System. The second data source may include a set of information regarding a liable third party, a settlement, or other identified payer/obligor. The information may indicate whether there exists a third party payer/obligor who could be called to account for all or any part of a patient's healthcare expenses.

At step 750, based on the information from the first data source, third party liability can be identified and/or assessed corresponding to the identified patient.

In some instances, a second set of third party liability information for the identified patient may be received at step 760, the second set being responsive to the identification and/or assessment previously made. The responsive information may be taken into account, in conjunction with the first set of third party liability information, the other information previously processed, and a second identification and/or assessment of third party liability may be generated and sent to the patient and/or healthcare provider. Additional data sources can be used in order to decrease the likelihood of redundant billing, and ensure that payers with higher reimbursement rates are billed.

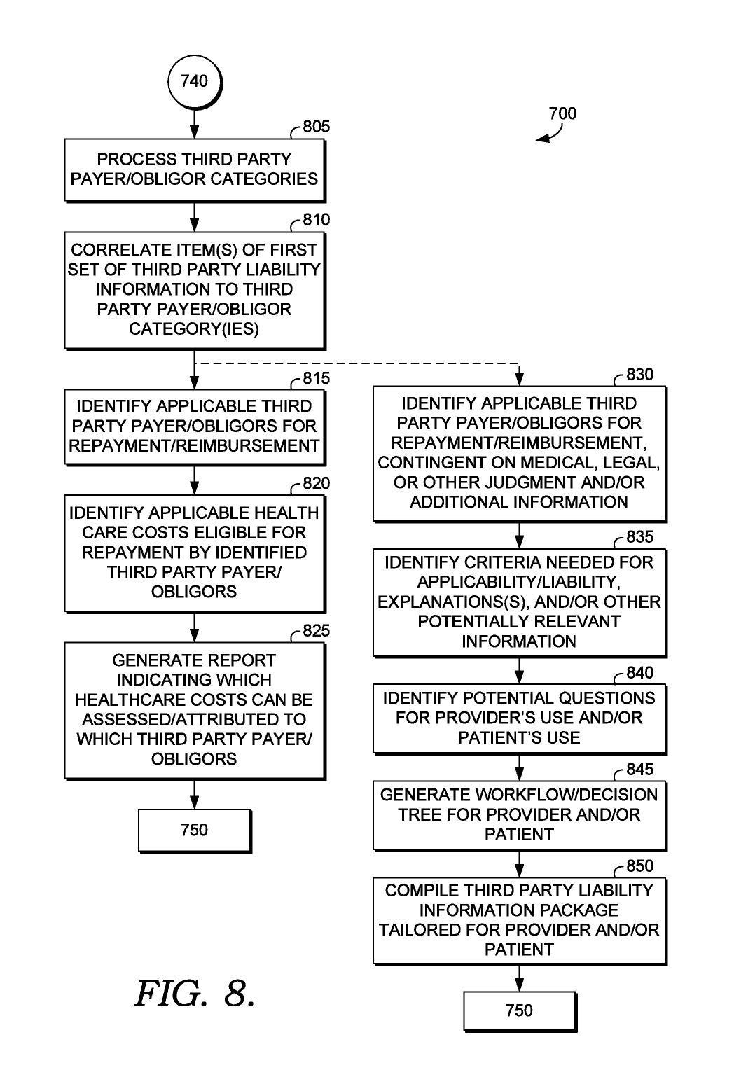

FIG. 8 illustrates a method 800 for generating third party liability information corresponding to the patient, in accordance with some embodiments of the present disclosure. Alternatively or additionally to third party payer/obligor information, some embodiments may operate with respect to judicial and/or legal settlement agreements or decisions and/or other forms of third party liability. Method 800 may begin by transitioning from step 740 in FIG. 7 to step 805, in which third party liability categories may be processed. Any possible third party payer/obligors identified earlier (e.g., in step 350) may be taken into account, and data regarding the third party liability information may be prepared for correlating to any third party liability information of the identified patient. Categorization based at least in part on the letter grade or alternate category assigned in step 345 may be taken into account. These categories may also be based on the scope of third party liability and/or third party reimbursement rates.

At step 810, item(s) of the first set of third party liability information may be correlated to third party liability identification and/or assessment of liability to determine which third party payers/obligors are applicable to the patient and which of these party(ies) may be applicable to the patient depending on the medical, legal, judicial, or other judgment of a healthcare provider tending to the patient and/or depending on additional information that is needed to make that determination. The healthcare provider's judgment might be relevant, for example, to whether or not two separate diagnoses are related to the same injury or accident, and the provider's judgment might be identified from patient encounter notes in a healthcare record for the patient, or might be solicited, for example, using a provider interface. At step 815, third party payers/obligors applicable to the patient may be identified. At step 820, healthcare costs to the patient that a third party obligor/payer are liable for may be identified. At step 825, a report indicating which healthcare costs to the patient a third party obligor/payer are liable for may be generated and/or sent to the healthcare provider's billing department.

At step 830, third parties whose liability is contingent on the medical, legal, judicial, or other judgment and/or for whom additional information is required to make a determination of liability may be identified. At step 835, criteria needed for applicability/eligibility/liability, explanation(s), and/or other potentially relevant information may be identified, generated, gathered, and/or organized. The third party payer/obligor may have reimbursement guidelines that might specify what qualifying information is needed in order to determine services and/or products for which the third party payer/obligor will provide payment.

At step 840, potential questions for the determination of third party liability for provider's use and/or patient's use may be identified, gathered, and or organized. At step 845, a workflow and/or decision tree for the provider and/or patient may be identified and/or generated. At step 850, a report tailored for the provider and/or patient may be compiled. Then, the process flow may transition to step 750 of FIG. 7.

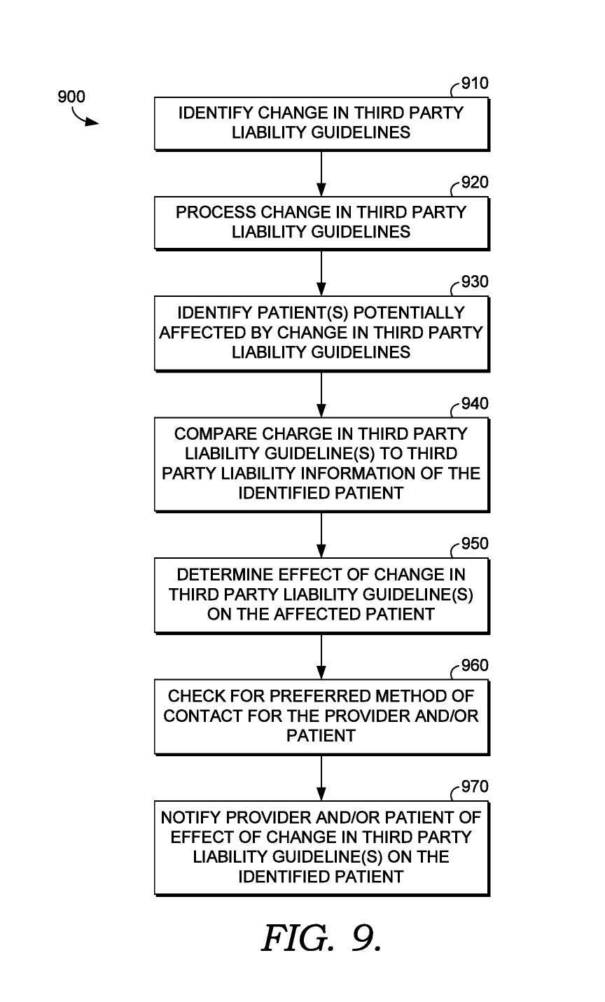

FIG. 9 illustrates an exemplary method 900 for handling changes in third party payer/obligor guidelines, in accordance with some embodiments of the present disclosure. Alternatively or additionally to third party payer/obligor identifications, some embodiments may provide such features with respect to settlement or other legal or judicial agreements or decisions.

One or more changes in third party liability guidelines may be identified at step 910. The identification may be way of linking to a site that provides updates on such changes, periodically crawling sites for updates and changes, and/or otherwise receiving notice of changes in the guidelines.

The changes in third party liability guidelines may be processed at step 920. The scope of the changes may be identified. As a non-limiting example, it may be determined that a certain insurance company has changed their reimbursement guidelines for certain types of patients, certain payers, etc. As another non-limiting example, it may be determined that the changes translate to greater or lesser thresholds for repayment applicable to certain patients and/or eligibility for cost-sharing by payers. Data, regarding details, scope, extent, and/or potential ramifications of the changes, may be prepared for correlating to the third party liability information of particular patients.

A patient potentially affected by the changes in third party liability guidelines or other changes may be identified in step 930. The identification of the patient may be based on a determination that one or more of the changes affect certain insurers, certain thresholds for liability, changes in legal or judicial framework, changes in laws or regulations, certain types of patients, certain payers, etc., and correlating those determinations with the patient.

Changes in third party liability guidelines may be compared to the third party liability information of the identified patient in step 940. The comparison may consider, for non-limiting example, the past identifications of third party payer/obligors, settlement offers, changes in insurer guidelines, etc., in order to correct any out-of-date information or identifications and/or assessments. The comparison may consider threshold conditions of the patient, such as age thresholds (e.g., a minor below a certain age may be incapable of negligence), cost thresholds (e.g., healthcare costs above or below a certain amount may not be covered by a third party insurer), and the like that may affect whether or when a third party will be liable for a patient's healthcare costs.

Effects of changes in third party liability guidelines on the identified patient may be determined in step 950. The effects may be revealed as a result of the previous comparisons. The effects may be compiled and formatted for communication to the patient and/or the healthcare provider. At step 960, there may be a check for a preferred method of contact for the provider and/or patient. A healthcare provider or patient, having previously set up an account, may have specified a preferred method, whether it be text, voice, e-mail, or paper mail. In that way, those that are affected by the changes may be promptly notified. At step 970, the patient and/or the patient's provider may be notified of the effects of changes in third party liability guidelines on the identified patient.

In various embodiments, one or more interfaces such as one or more of the following: a patient portal, a healthcare provider portal, a healthcare payer portal, and/or another portal may be used as a means of third party liability information access. In some embodiments, a patient may be asked for permission to share third party liability information when the patient uses the patient portal, a healthcare provider portal, a healthcare payer portal, and/or another portal. In some embodiments, patient permission may be granted to one or more healthcare providers, and such permission to a healthcare provider may be extended to one or more employees or agents of the healthcare provider.

In some embodiments, a patient may be directly contacted for such permission, for example, in conjunction with an explanation of a patient's legal rights. In various embodiments, a patient may opt out of sharing at any moment in time, a patient may be permitted to restrict the sharing of third party liability information in various ways, a patient may restrict the duration for which permission is granted, and/or permission may be granted until revoked.

In some embodiments, a workflow may include acquisition of third party liability information external to the system. The workflow could include generating documents based at least in part on the third party liability information. These documents may be useful in obtaining information from or transferring information to or between relevant entities who have elected not to participate in the system, or who use paper-based rather than electronic records. Examples of such entities might include patients who lack computer skills or network access, or providers outside of the system, such as attorneys' offices or courts. The workflow could include transferring documents to or between one or more of a healthcare provider, a healthcare payer, an attorney, a court, a liable third party, and/or a patient. In some embodiments, transferring documents may be in conjunction with face-to-face contact with a patient. For example, documents may be presented to and/or reviewed with the patient during a medical appointment. If the document is soliciting information, the information may be entered directly into the system by a healthcare provider or assistant, or may be recorded on the document for later entry into the system by a system user.

In some embodiments, if a patient has an existing account with one or more of a healthcare payer and/or provider, information needed to access the account may be acquired. In some embodiments, if a patient does not have an existing account with one or more of a healthcare payer and/or provider, patient permission to create an account on behalf of the patient may be acquired. In some embodiments, account handling may be automated. For example, patient consent and/or patient preferences may be solicited via a patient interface. For patients with limited computer skills and/or access to the network, patient input may be solicited using a device at healthcare facility, such as a computer or kiosk, which might or might not be dedicated to patient use, such as patient check-in for a medical appointment. Any one or combination of embodiments disclosed herein may be automated in whole or in part.

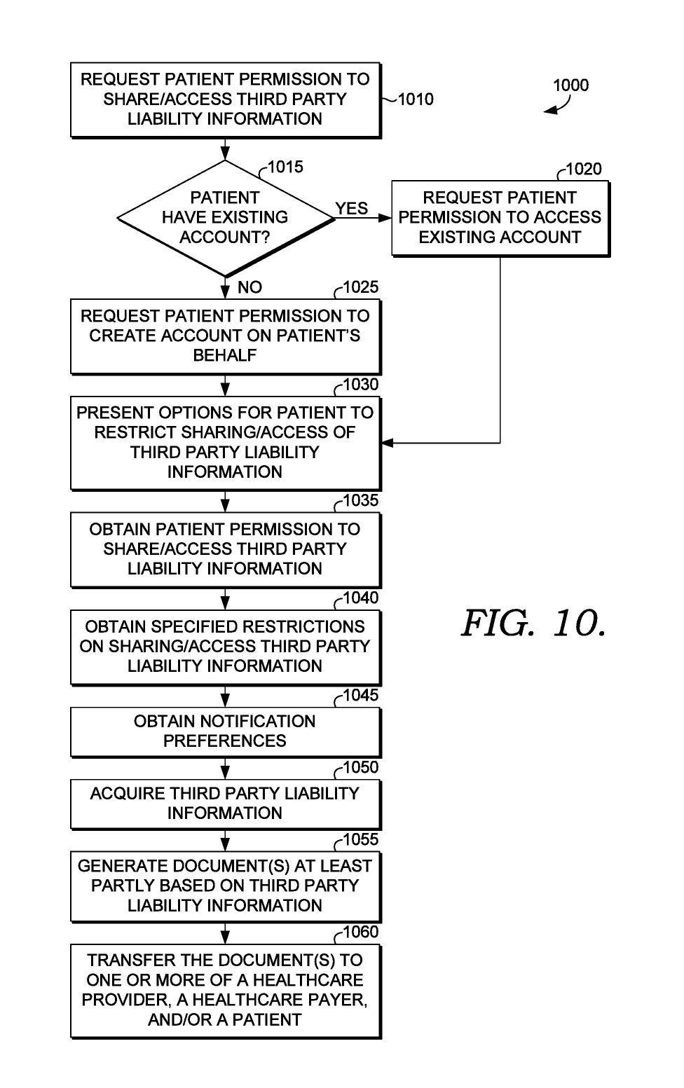

FIG. 10 illustrates an exemplary method 1000 of facilitating acquisition of patient consent, preferences, and information in accordance with some embodiments of the present disclosure. Patient permission to share and/or access third party liability information may be requested by the healthcare provider at step 1010. This request may be made electronically or through other means of obtaining consent. At step 1015, it may be determined whether the patient has an existing account. In the case that the patient has an existing account, patient permission to access the existing account may be requested by the healthcare provider at step 1020. In the case that the patient does not have an existing account, patient permission to create account on patient's behalf may be requested by the healthcare provider at step 1025.

Options for allowing the patient to restrict sharing and/or access of third party liability by the healthcare provider may be presented at step 1030. Patient permission to share and/or access third party liability information may be obtained by the healthcare provider at step 1035. Specified restrictions on sharing and/or accessing healthcare information may be obtained by the healthcare provider at step 1040. Patient notification preferences may be obtained by the healthcare provider at step 1045.

Third party liability information may be acquired by the healthcare provider at step 1050, as reported by the third party. For example, the permission obtained at step 1035 may relate to access to an insurance provider's online account information for the patient. If the patient does not have online access to the patient's insurance information, the permission requested may be to create an online account or initiate online access to an account for the patient. The system may then download a patient's records from the insurance provider's online account.

Document(s) at least in part based on the third party liability information may be generated at step 1055. Non-limiting examples include documents listing the identity and billing information of third party payers/obligors and documents evidencing a judicial settlement between the patient and a third party. The document(s) may be transferred to one or more of the following: a healthcare provider, a healthcare payer, and/or a patient, in step 1060.

In some embodiments, third party liability may be processed, e.g., re-assessed or re-evaluated, on a periodic basis, such as a monthly basis, or on any suitable time basis. New third party liability information may arise from time to time, for example, if a new claim was filed, or a judgment or settlement was recorded in a court case. New third party liability information may be identified based on the differential with respect to the last set of third party liability information acquired from a particular source. In some embodiments, a time basis may depend on proximity to a date of eligibility for a service, such as scheduled medical screenings or follow-up appointments, and/or date of compliance, such as compliance with regulatory or contractual billing guidelines for submitting bills or requests for reimbursement for a patient's medical care.

In some embodiments, a patient may be an integral part of processing third party liability information. Some embodiments may monitor third party liability information and notify a patient of actions taken and/or status with regard to the patient's third party liability information. Some embodiments may allow the patient to review the patient's third party liability information and actions taken and/or status of the patient's third party liability information. Some embodiments may allow the patient to object to, request corrections of, or otherwise address certain actions taken or resolve any issues that arise.

In some embodiments, data from Medicare's Common Working File may be an integral part of processing third party liability information. Some embodiments may monitor third party liability information obtained from Medicare s Common Working File and/or other Medicare database(s), Medicaid database(s), document(s), record(s), etc., and notify the healthcare provider of new information contained therein.

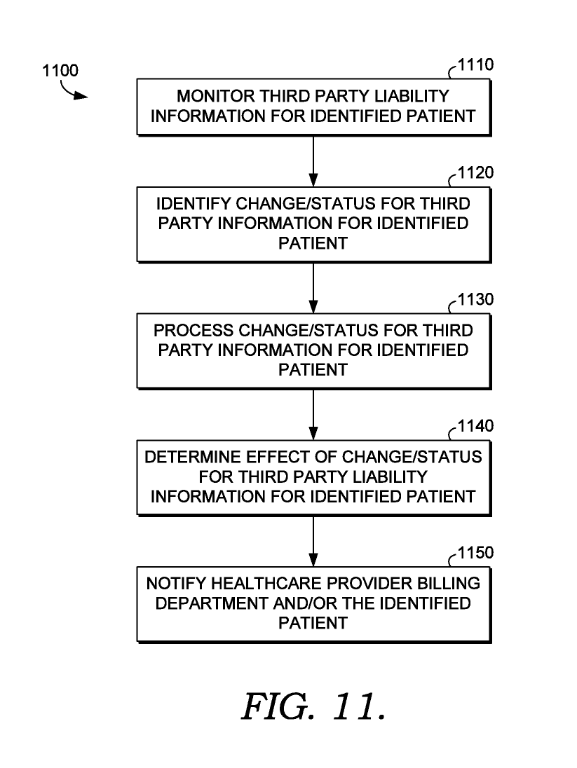

FIG. 11 illustrates an exemplary method 1100 of facilitating monitoring of a patient's third party liability information in accordance with some embodiments of the present disclosure. New or updated third party liability information for an identified patient may be monitored by the Health Information Handing System in step 1110. A change and/or status of the third party liability information for the identified patient may be identified in step 1120. The change and/or status of the third party liability information for the identified patient may be processed at step 1130, e.g., to import the new information into the system, as by downloading electronic data or generating a request for information stored in non-electronic form or otherwise not electronically available to the system.

One or more effects of the change of status for the identified patient may be determined in step 1140. As a non-limiting example, a patient's total healthcare costs may rise above a certain threshold level which could trigger a repayment obligation on the part of a third party, or a patient may abandon a legal claim against a third party and absolve them of liability. The new or changed information may also be weighted, as described above. The healthcare provider and/or the identified patient may be notified of the effect of the change and/or status for the third party liability information for the identified patient in step 1150. The Health Information Handling System may be updated to reflect any changes, and that information may be stored in one or more memory(ies) or repository(ies).

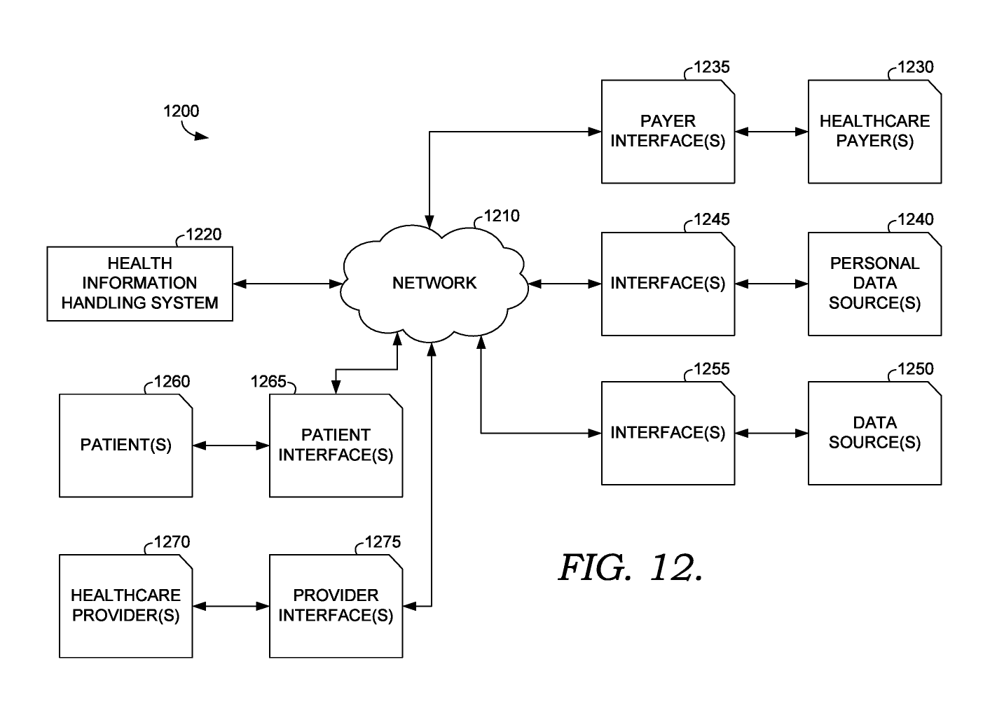

FIG. 12 shows a high-level block diagram of an exemplary computing system 1200 in accordance with aspects of the present disclosure. The network 1210 may be any suitable means to facilitate data transfer in the system. Non-limiting examples include utilizing one or more of the following: the Internet, a wide area network (WAN), a local area network (LAN), a wireless local area network (WLAN), intranet, a metropolitan area network (MAN), a cellular network, such as 5G, 4G, 3G, GSM, etc., another wireless network, a gateway, and/or any other appropriate architecture or system that facilitates the communication of signals, data, and or messages. The network may transmit data using any suitable wireless or wired communication protocol. The network and its various components may be implemented using hardware, software, and communications media such as wires, optical fibers, microwaves, radio waves, and other electromagnetic and/or optical carriers, as well as any combination of the foregoing.

The health information handling system 1220 may be communicatively coupled or couplable to the network 1210. The health information handling system 1120 may include any device or set of devices configured to compute, process, store, display, handle, and/or use any form of information and/or data suitable for embodiments described herein.

For example, the health information handing system 1220 could include a single computer server, or multiple computing devices which may be implemented in or with a distributed computing and/or clouding computing environment with a plurality of serves and cloud-implemented resources. Thus, a health information handling system 1220 may include one or more servers. The health information handling system 1220 may include one or more processing resources communicatively coupled to one or more storage media, random access memory (RAM), read-only memory (ROM), flash memory, and/or other types of memory. The health information system may include any one or combination of various input and output devices (I/O) devices, databases, network ports, and display devices.

Healthcare payer(s) 1230 may include any individual, entity, organization, or institution that provides for payment related to healthcare, healthcare services, and/or claims for healthcare services, and/or that administers insurance and/or health-related benefits. Non-limiting examples of healthcare payers 1230 include government agencies/programs (e.g. Medicare, Medicaid), private insurance companies, a health maintenance organization (HMO), a preferred provider organization (PPO), an organization that may be contracted by said examples, a financial institution responsible for handling the affairs or obligations of an individual, etc.

Healthcare payer information may include one or more of a database, any repository of data in any suitable form, a website, and/or a server. In some aspects, the healthcare payer information may be a source of health information and/or payer benefit information and/or third party liability information relating to patients. By way of example without limitation, Medicare, a government entity, as a healthcare payer, who could be a user that receives information whether through the network or otherwise, may provide information to the health information handling system and third party liability, and/or may have access to records, databases, and/or other repositories containing information about third party liability. Medicare's information repositories such as Medicare's Common Working File may be electronically linked or otherwise in electronic communication to the health information handing system such that the repositories may correspond to the healthcare payer in some embodiments.

Payer interfaces 1235 include, without limitation, a web interface allowing for transfer of and access to one or more of biographical information, demographical information, medical information, medical conditions, care provided, preventive/curative/palliative/other care recommendations, preventive/curative/palliative/other care eligibility, payer coverage, regulatory information, third party liability information, etc. These payer interfaces 1235 may be connected to the health information handling system 1220 via the network 1210 so that patient-related information retained by the personal data source(s) 1240 may be accessed by/transferred to the health information handing system 1220.

Interfaces 1245 may include any suitable input/output module or other system/device operable to serve as an interface between the personal data sources 1240 and any other data sources 1250 and the network 1210. The interfaces 1245 may facilitate communication over the network 1210 using any suitable transmission protocol and/or standard. For example, the health information handling system 1220 may include and/or provide one or more of the interfaces 1245 by making available one or more of the following: a website, web portal, web application, mobile application, enterprise software, and/or any suitable application software.

These data sources 1250 may contain confidential health and third party liability information for patients. Confidential health information may include, without limitation, any information on one or more of the following: health conditions, medical conditions, characterizations, assessments, test results, and/or various metrics for specific patients, biographical/demographical information for specific patients, prescription information for specific patients, care services provided to specific patients, accident/incident location information, third party payer information, and/or eligibility of specific patients for health care services.

In some embodiments, the patient 1260, or legal representatives thereof, may access the health information handing system 1220 through one or more patient interface(s) 1265. As depicted, the one or more patient interfaces 1265 may be communicatively coupled or couplable to the network 1210. By way of example, and without limitation, a patient interface 1265 may include a web portal accessible from the network 1210, and the health information handing system 1220 may include, provide, and/or facilitate providing an application to the patient 1260. In some embodiments, the health information handing system 1220 may include and/or provide the patient interface 1265, for example, by making available one or more of the following: a website, a web page, a web portal, a web application, a mobile application, enterprise software, and/or any suitable application software.

The patient interface(s) 1265 may include, for example and without limitation, a web interface allowing for transfer of and access to one or more of the following: biographical information, demographical information, medical information, medical conditions, care provided, preventive/curative/palliative/other care eligibility, payer cover, third party liability information, regulatory information, etc. In some aspects, the patient 1260 may access the patient's aggregated health information serviced by the health information handing system 1220. First time users, or legal representatives, might have to set up an account, along with authentication information. Subsequent to authentication, a patient 1260, or a legal representative, may have access to confidential health and liability information for the patient 1260.

Some embodiments may allow the patient 1260 to track the patient's health and payer liability information, consolidated from various sources into one place, via the patient interface 1265. The patient 1260 may be able to see who is liable for their medical coverage, see who is currently being billed for their services, see if a liable party is not being billed, and see the extent of third party liability from zero-liability to some liability to full liability. The patient 1260 may be able to see third party liability information that may be applicable but contingent on further information to be provided by the patient 1260 and/or the patient's legal representative or other party with knowledge of third party liability.

The healthcare provider 1270 may be a user of the health information handling system 1220, and/or a source of health information about patients. Non-limiting examples include a healthcare provider disseminating information to the health information handing system 1220 about patients and/or providing records, documents, or access to other repositories containing patient information.

The healthcare provider interface(s) 1275 may include without limitation a web interface allowing for transfer of and access to one or more of the following: biographical information, demographical information, preventive/curative/palliative/other care eligibility, payer coverage, third party liability, regulatory information, etc. Healthcare providers 1270 may have website/portals giving access to such information. The healthcare provider interface 1275 may include any suitable input/output module or other system/device operable to serve as an interface between the healthcare provider 1270 and the network 1210. The healthcare provider interface 1275 may facilitate communication over the network 1210 using any suitable transmission protocol and/or standard.

Some embodiments allow the healthcare provider 1270 to track patient's health information and third party liability information, consolidated into one place. The healthcare provider 1270 may be able to see potential third party payers/obligors, and see the extent of liability from zero-liability to some liability to full liability. The healthcare provider 1270 may be able to see what third party liability or obligations may be applicable contingent upon further information provided by the patient 1260 and/or the medical judgment of the healthcare provider 1270. The healthcare provider 1270 may be given explanation(s), non-limiting examples including: the location of an incident, third party insurance information, third party settlement information, third party promises, third party obligations, additional payer information, any further information needed, other potentially relevant information, etc., and combinations thereof. In some aspects, where additional information is required or helpful, a workflow can be specified for the healthcare provider 1270 that could include a decision tree to gather information used in determining payer liability. The workflow may include any one or combination of a graphical decision tree, a textual decision tree, a series of prompts configured to walk to the healthcare provider 1270 through a decision tree, a flowchart, and instructional narrative, a list, and/or the like. The healthcare provider 1270 may have the option to print the workflow. The third party liability guidelines may be provided along with information needed to comply with reimbursement eligibility. Different payers can have different reimbursement guidelines and eligibility such that each patient/healthcare provider might have a customized interaction with each payer/obligor. Indications of the customized information may be provided to the healthcare provider 1270. Any combination of the foregoing may be provided to the healthcare provider 1270 though a single provider interface 1275.

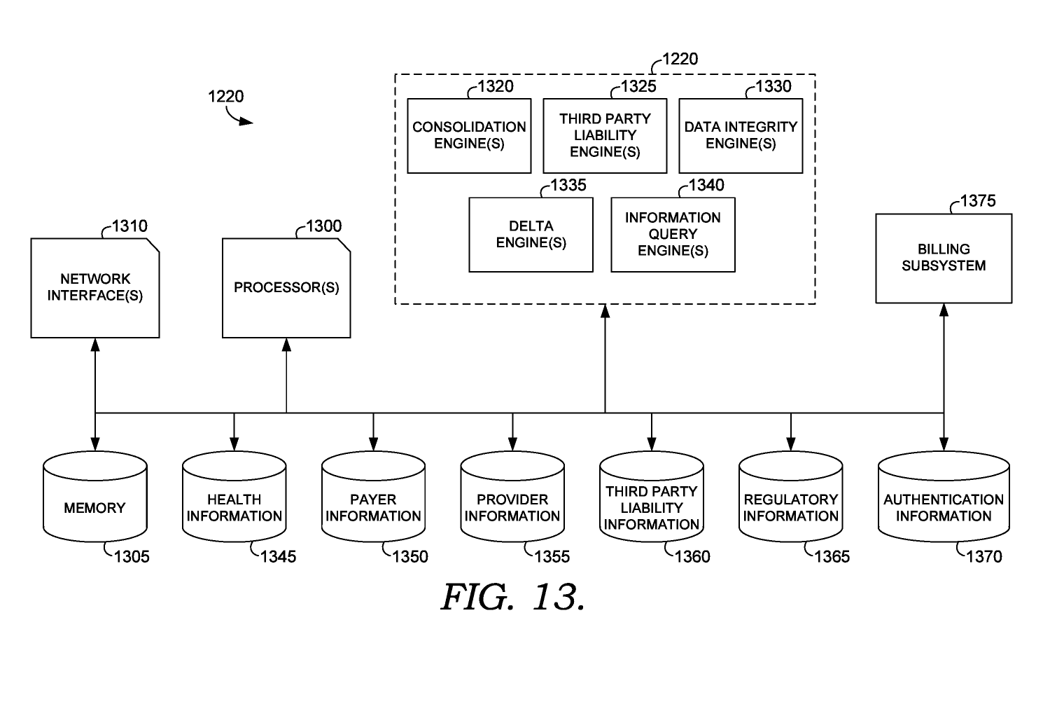

FIG. 13 shows a high-level block diagram of one exemplary embodiment of the health information handing system in accordance with some aspects of the present disclosure.

The health information handling system 1220 may include one or more processors 1300 which are communicatively coupled with one or more memories 1305 and/or electronic databases stored in the one or more memories 1305. The consolidation engine and/or other data may be stored within memory 1305 or databases for access by the health information handing system 1220. The health information handling system 1220 may include one or more network interfaces 1310 (such as one or more of interfaces 1235, 1245, 1255, 1265, and/or 1275, as shown in FIG. 12) communicatively coupled to processors 1300. The network interface(s) 1310 may include any suitable input/output module or other system/device operable to serve as an interface between one or more components of the health information handing system 1220 and the network. The health information handling system 1220 may use the network interfaces 1310 to communicate over the network using any suitable transmission protocol or standard.

Various embodiments of the health information handling system 1220 may also include one or more engines to implement any combination or sub-combination of features or embodiments described in the present disclosure. In various embodiments, the engines may include one or more of consolidation engine(s) 1320, third party liability engine(s) 1325, data integrity engine(s) 1330, delta engine(s) 1335, and/or information query engine(s) 1340. While the engines are depicted separately, it should be appreciated that in various embodiments the one or more engines may be a consolidation engine 1320, a third party liability engine 1325, a data integrity engine 1330, a delta engine 1335, and/or an information query engine 1340, collectively and/or integrally as a single engine 1315. These engines may be stored in memory and may include one or more processors, for receiving and processing data requests. The engines may be configured to perform any of the steps of the methods described in the present disclosure.

By way of example without limitation, the consolidation engine(s) 1320, with one or more of processors 1300, may utilize one or more of the network interfaces 1310 to access one or more of the healthcare provider's 1270, the healthcare payer(s) 1230, the personal data sources 1240, and/or the other data sources 1250, through the network 1210, as shown in FIG. 12. The health information handling system 1220 may push and/or pull confidential health information from these entities in any suitable way. The consolidation engine 1320 could process data pulled and/or pushed from the entity. In some instance, health information could be pre-loaded and/or directly transferred to the health information handling system 1220 (e.g., via a storage medium) in addition to or in lieu of transferring data via the network. The consolidated data may be retained in one or more of the memories 1305.

The consolidation engine 1320 may accord a measure of reliability, consistency, comprehensiveness, thoroughness, and/or accuracy to the confidential health and/or payer/third party liability information that corresponds to a specific patient. All of the specific patient's health information and/or payer/third party liability information may be consolidated into one place. The consolidation engine 1320 may access manifold sets of confidential health information and/or payer/third party liability information that correspond to a specific patient. For instance, different sets of information may come from different sources. Different sets of information could come from the same source, for example, by way of one or more updates to information previously provided by a particular source for a particular patient. The consolidation engine may consolidate the sets of confidential health information and or payer/third party liability information for the particular patient. Having determined the sets correspond to an identified patient, the consolidation engine may form a composite set of confidential health information and/or payer/third party liability information. The consolidation may include organizing, categorizing, qualifying, and/or comparing the sets of information; detecting, identifying, and/or handling errors/discrepancies; and/or otherwise processing the sets of confidential health information and/or payer/third party liability information for the identified patient. Thus, the health information and/or payer/third party liability information may be automatically organized into easy-to-understand categories, potentially by categorizing previously uncategorized data and/or re-categorizing data that was previously categorized for a different purpose, erroneously categorized, could not be categorized (for example, because of non-standardized data or file formats), or using categories that are not useful in the practice of the methods described in this disclosure. The consolidation engine may store the composite set of confidential health information and/or payer/third party liability information for the identified patient in memory or a database. For example, the composite set of third party liability information may be retained in one or more third party liability information repositories.

The consolidation engine 1320 may acquire and store authentication information in one or more authentication repositories. The authentication information may be for a user that is approved for access to at least part of the composite set of confidential health information and/or payer/third party liability information for the identified patient. For example, the user, who may be the healthcare provider, may use one of the interfaces to seek access to the patient's third party liability information. The user may provide a set of credentials in order to gain access. The authentication information, which may be of any suitable form and content, may be retrieved and used to check the credentials provided. Pursuant to authentication, the user may have access to some or all of the composite set of information corresponding to the identified patient. The access could be limited to any suitable confines to maintain privacy.

In some embodiments, the third party liability engine 1325, with one or more processors, may be configured to provide a provider with third party liability/settlement information corresponding to a patient(s). The third party liability engine 1325 may identify a patient. In some embodiments, the identification of a patient may be initiated by another user such as the healthcare provider. The third party liability engine 1325 may derive third party liability/settlement information for the identified patient for a source, as a non-limiting example, by pulling and/or pushing third party liability information from one or more of the following: the healthcare providers 1270, the healthcare payers 1230, the personal data sources 1240, and/or any other data sources 1250—or by processing preloaded and/or otherwise directly transferred third party liability/settlement information. The third party liability engine 1325 may access third party liability information—as a non-limiting example, settlement information stored in one or more third party liability repositories.

The third party liability engine 1325 may access one or more sources to identify a potentially liable third party. In so doing, the third party liability engine 1325 may access one or a combination of health information repositories, the payer information repositories, the provider information repositories, the third party liability information repositories, the regulation repositories, and/or the authentication information repositories. Accordingly, in various embodiments, the set of criteria may be based on various items of information.

By way of example, without limitation, in some embodiments, the criteria may be based in part on the information which may be stored in the payer information repositories. Thus, criteria could correspond to third party liability for one or more of the following services: preventive, curative, palliative, and/or other care services, and may indicate whether one or more of these services are eligible for payment by liable third parties. In some embodiments, the criteria may be based at least partially on that which may be stored in the provider information repositories. Thus, criteria could be based on the location of an incident, availability of insurance coverage, etc. In some embodiments, the criteria may be based at least partially on that which may be stored in the third party liability information repositories. Thus, criteria could be based on data made available. As a non-limiting example, by information obtained through information and/or data provided by Medicare's Common Working File, Medicare, Medicaid, etc. Criteria could be based in part on liability or settlement information (e.g. derived from any suitable judicial entity, information provided by the patient/the patient's legal representative, a third party, and/or the like) that may identify a potentially liable third party. In some embodiments, the criteria may be based at least partially on what may be stored in the regulation information repositories. Thus, criteria could be based at least partially on regulations issued by a government authority, rules/guidelines for implementing regulations, and/or the like. In some embodiments, the criteria may be based at least partially on that which may be stored in the authentication information repositories. Thus, criteria could be based in part on retractions of access to information pertaining to a patient.

The third party liability engine 1325 may take into account the set of criteria, and may generate a specific third party or third parties liable for payment corresponding to the identified patient. The third party liability engine 1325 may correlate confidential health information and/or liability information to the set of criteria in view of the patient's healthcare costs. Items of confidential healthcare information of the patient may be compared to details of third parties liable for payment to determine which third parties are liable for which costs (e.g. the insurer of a venue where a slip-and-fall occurred) and which third parties may be liable for payment depending on judicial determination (e.g. awaiting trial or settlement) and/or depending on additional information that is needed to make that determination.

The payer and/or third party may have reimbursement guidelines that might specify what qualifying information is needed in order to determine services for which the payer/and or third party will provide payment. Where additional information is required, a workflow can be specified for a healthcare provider that could include a decision tree to gather information used in diagnosis with areas of medical and/or legal judgment left to the provider. Any unanswered questions may be fed back to the third party liability engine. The preventive, curative, palliative, and/or other care guidelines may be provided along with the information needed to comply with reimbursement eligibility. Different payers and/or third parties can have different preventive, curative, palliative, and/or other guidelines along with reimbursement eligibility such that each patient might have a customized interaction with the provider.

In some embodiments, the data integrity engine 1330 with one or more processors, may check health information to ensure the quality of data underlying the identification of a third party payer for a particular patient. The data integrity engine 1330 may assess each piece of information underlying the identification of a potentially liable third party and may assign a weight to the information according to a score. Any suitable scoring system may be used. Missing information, for example could have a lower score than non-missing information; and the missing information could be scored even lower, the more important the information is to the identification of a third party. Information may be weighted according to the source. As a non-limiting example, in some instances, information gathered from Medicaid might be weighted higher or lower relative to information gathered from a patient. Scoring recommendations based on the information based upon the underlying reliability of information may avoid redundant, potentially harmful and/or unnecessary billing of payers. The data integrity engine 1330 may utilize one or more network interfaces 1310 to convey the results of the third party liability scoring to a user.

In some embodiments, the data integrity engine 1330 may examine items of information and assign scores according to how important such informative is to attributing third party liability generally. In some embodiments, the data integrity engine 1330 may adjust scoring of information in view of specific third party liability information for a given patient. In some embodiments, the data integrity engine 1330 may examine items of information in view of specific information regarding a third party payer/obligator upfront, thereby rendering subsequent readjustment unnecessary.

Based on the scoring, possible follow-up questions and/or prompting for further information and/or clarifying information may be identified, generated, and/or provided by the data integrity engine 1330. Accordingly, third party payers/obligors can be identified more reliably with the provider asking possible follow-up questions and/or prompting for an account to link for more missing health and/or liability information. The data integrity engine 1330 may utilize the network interface(s) to convey the results of the third party payer/obligor identification scoring to a user.

In some embodiments, the delta engine(s) 1335, with one or more processor(s), may be configured to handle changes in third party liability. The delta engine 1335 may recognize changes in third party liability guidelines (e.g., those implemented by law/regulation). In some embodiments, the delta engine 1335 may be similarly directed to curative, related, and/or other care guidelines issued by any suitable entity, such as government, regulator, and/or recommendation entity. The health information handling system 1220 may be linked to a site that provides updates on such changes, may periodically crawl for updates and changes, and/or may otherwise receive notice of changes in the guidelines. The delta engine 1335 may process the changes to identify exactly what has changed, the scope of the changes, and potential ramifications. The delta engine 1335 may prepare data, regarding details, scope, extent, and/or potential ramifications of the changes, for correlating to the third party liability for healthcare costs of particular patients. As a non-limiting example, it may be determined that the changes affect limitations on liability for landowners regarding premises liability; it may also be determined that the changes translate to greater or lesser thresholds for liability to be attributed to a third party.

The delta engine 1335 may identify patients potentially affected by the changes. The identification of the patient may be based on the determinations that the changes affect certain third party obligations, liability, settlements, certain types of patients, certain payers, etc. The delta engine 1335 may compare the data regarding the changes to confidential health and/or settlement/third party liability information of the identified patient. Then the delta engine 1335 may notify providers and/or patients of those changes. The delta engine 1335 may check for the preferred manner of contact stored in the information handling system 1220 for the provider and/or patient. Any suitable means of notification may be employed. For example, text, voice, e-mail, and/or paper mail notifications could be sent to those affected by revisions. The notification could include a link or other communication reference referring back to a provider/payer site and/or health information portal provided by the health information handling system to get the confidential information about the changes in third party liability/obligations/settlements on the identified patient.

In some embodiments, the information query engine(s) 1340, with one or more processors, may be configured to handle searching of one or more information repositories. Other engines may include and/or utilize the information query engine 1340 in various embodiments. The searching may be in response to information received over the network 1210 from a user. The information query engine 1340 may allow for user identification of various suitable search criteria, according to various embodiments. By way of example, without limitation, the information query engine 1340 may receive a query from a provider, payer, or patient, where the query is transferred over the network to the health information handling system 1220. In some embodiments, the query may have a packetized structure according to a packet protocol, and may include one or more search terms. Responsive to the query, the information query engine 1340 may search, retrieve, modify, and/or cause transfer of particular information from one or more information repositories.

One or more health information repositories may retain any health information suitable for embodiments of this disclosure. The health information repositories 1345 may include database(s), database management system(s), memory, and/or server(s) to facilitate management/transfer/provision of health information, and the like. The repositories may retain confidential health information of particular patients. The confidential health information may include, without limitation, any information on one or more of the following: health conditions, medical conditions, characterizations, assessments, test results, and/or various metrics for specific patients, prescription information for specific patients, care services provided to specific patients, hospital/ambulatory incident reports for specific patients. As noted, the confidential health information may be aggregated from any one or combination of the following: healthcare providers, healthcare payers, third party obligors, the personal data sources, and/or the other data sources.

One or more payer information repositories may retain any suitable information related to healthcare payers. The payer information 1350 may include, without limitation, payer identification information, third party payer identification and/or information, third party obligor identification and/or information, settlement information, payer policy information, coverage/benefits guidelines/rules for services, healthcare plans, explanations of benefits, in-network/preferred provider information, and the like. The payer information could indicate qualifying information necessary for determinations of coverage eligibility or third party liability for healthcare services. The payer information could include one or more of the foregoing items with respect to particular patients.

One or more provider information repositories may retain any suitable information related to healthcare providers. The provider information 1355 may include, without limitation, provider identification information, provider location, amenities offered by providers, provider schedule information, technology offered by providers, billing/payer information, third party obligor information, third party insurer information, in-network/preferred provider information, settlement information, advertising information, provider billing information, reviews of providers, provider feedback and the like. The provider information 1355 could, for example, be used to provide third party insurance information to billing departments of healthcare providers using the system.

One or more third party liability information repositories may retain any suitable Information related to third party insurers/obligors/settlements/decisions for any type of healthcare afforded to a patient, such as curative care and/or palliative care. The third party liability information 1360 may include without limitation: information on settlements, third party insurers, third party obligors, pending litigation, potential third party payers, legal or judicial decisions or rulings, arbitration awards, administrative decisions or rulings, promises or statements made to patients and/or their legal representatives or other party. The third party liability information 1360 may be organized for correlation to confidential health information of particular patients. The third party liability information 1360 may include, but is not limited to, locations of incidents, promises or statements made, the existence or absence of third-party insurance, admissions of liability, etc., for filtering to identify pertinent third party payers/obligors. Third party obligations may be characterized as to whether they are routine or require additional information. Those obligations categorized as routine might be directed to an automated workflow for directing a bill or other payment request to the responsible third party. Those obligations categorized as requiring additional information might be directed to a workflow comprising automated and/or user-facilitated information requests and information gathering, while the bill or other payment request is withheld from submission to one or more payers until the additional information is received and analyzed. As a non-limiting example, additional information from a doctor or patient may be required, or there may be a judicial action pending which could impact third party liability. For routine payments, (e.g. payments from a third party insurer where there is no question of responsibility, such as an open question of fault or minimum or maximum thresholds of liability), payment and billing may be organized for ready correlation and billing of the appropriate third party. It would not, for example, be routine to send bills to an insurer when the policy limit has been exceeded. For third party obligations requiring additional judgment, information required/relevant may be likewise identified. In certain circumstances, previously prepared workflows may be specified for a provider as guidance for third party liability determinations. A workflow could include a decision tree to gather information used in assessing liability with areas of medical or legal judgment left to the provider.

One or more regulation information repositories may retain any suitable information related to regulation of health information, liability, and preventive/curative/palliative/other care. The regulatory information 1365 may specify the regulatory rules for controlling health information from health entities and patients. The regulatory information 1365 may include without limitations regulations issued by a governmental authority, rules/guidelines for implementing regulations, and/or the like. For instance, Medicare may promulgate regulations relating to their coverage of certain medical procedures. The regulatory information 1365 may include information relating to HIPAA regulatory policies, procedures, and guidelines for controlling and maintaining privacy/security of confidential health information.

One or more authentication information repositories may contain suitable authentication information to facilitate privacy and security for the system. The authentication information 1370 may include information and include information to check credentials of a patient, a legal representative, a healthcare provider, and/or a healthcare payer that may make use of their corresponding interfaces to seek access to a patient's confidential health information and/or other system-provided features. The authentication information 1370 may be used to restrict the access granted to a certain set of information. For example, access may be restricted to information pertaining to an identified patient; and access may be further restricted to a subset of such information as appropriate. A patient may only be provided access to his or her own patient information. A healthcare provider may have access to the information of several patients serviced by the healthcare provider (assuming that the patients have provided consent to allow the healthcare provider to access their patient information).

Any one or combination of the health information repositories, the payer information repositories, the provider information repositories, the third party liability information repositories, the regulation information repositories, and/or the authentication information repositories may include database(s), database management system(s), memory, server(s) to facilitate management/provision transfer of health information and the like. It should be appreciated that information in the corresponding repositories may be stored elsewhere and in other ways, or may not be stored, depending on the implementations chosen. Likewise, while various segregations of information corresponding to the repositories are provided herein, it should be appreciated that such examples are non-limiting, and some or all of the information may be handled in any suitable manner.

The health information handling system 1220 may include a billing subsystem 1375. The billing subsystem 1375 may handle billing aspects of accounts for the costs of preventive/curative/palliative/other care services. The billing subsystem 1375 may account for cost-sharing of services rendered or recommended. In some instances, multiple payers may be involved in covering a single curative care service. The billing subsystem 1375 may facilitate/coordinate the cost-sharing in such situations. Certain services can be mandated by law to be covered by insurers/plans in whole or in part. The billing subsystem 1375 may take such considerations into account. With payers, providers, and/or patients as users, breakdowns of coverage and cost sharing may be provided.

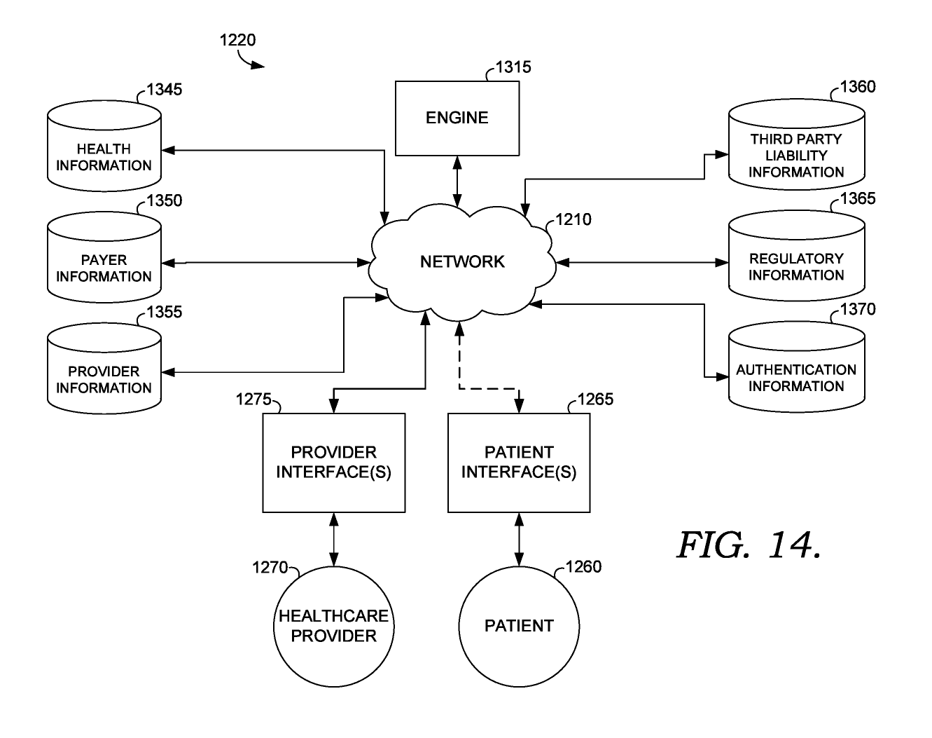

FIG. 14 is a block diagram of a system, in accordance with aspects of the present disclosure. The system may correspond to systems previously discussed, with FIG. 14 emphasizing possible interactions between the healthcare provider and/or the patient.

The healthcare provider 1270 or the patient 1260 may log into the system as a user via the provider interface 1275 or patient interface 1260, respectively. As noted previously, the interfaces may each include a mobile computing device or any other computing device. In some embodiments, one or both of the interfaces may include a mobile application installed on a mobile computing device for the patient/provider to use. The mobile application may be available for download, as a non-limiting example, from the health information handling system 1220. In some embodiments, one or both of the interfaces may include a web page, web portal, a web application, enterprise software, and/or any suitable application software for the provider/patient to use.

In the case of the healthcare provider 1270 or patient 1260 having previously set up an account, the user's credentials provided with login may be authenticated according to authentication information 1370 previously stored in the authentication information repository. Responsive to a login attempt the authentication information repository, which could correspond to one or more dedicated or shared servers (in some embodiments) may facilitate access to the authentication information 1370 previously stored for the particular patient. The engine(s) 1315 could facilitate authentication in conjunction with the authentication information repository.

As a consequence of authentication, the user may have access to certain confidential health and/or third party liability information. In the case of the healthcare provider 1270, the accessible confidential health and/or third party liability information may be relegated to a set of one or more patients previously identified as under the provider's care, identified as part of the authentication or subsequently identified by the provider. The healthcare provider 1270 could be allowed to select a particular patient from a list of patients or input an identifier for the particular patient. In the case of the patient 1260, the accessible confidential health and/or liability information may be relegated to only the patient's information.

Subsequent to the identification of the particular patient, the interface may present the patient's information. The patient's information can be arranged in any suitable way. For example, the patient's information can be arranged in the manner of a dashboard such that the provider may have a global view of the patient's information and/or categories of patient information. The health information 1345 and/or third party liability information 1360 may have been consolidated by the engines from various sources and automatically categorized into easy-to-understand categories. Items of information for selection by the healthcare provider 1270 may include, without limitation, biographical information, demographic information, medical information, medical conditions, care provided, preventive/curative/palliative/other care information, payer information, settlement information, third party liability information, payer coverage, regulatory information, etc. Dashboard items corresponding to such information may be categorized in any appropriate manner for ease of access and efficacy of presentation. The provider could be able to select and view a report of certain health information on the patient. For instance, the user could view the patient's medical conditions information retained in the system. Responsive to user selection, the third party liability repository, which in some embodiments could correspond to one or more dedicated or shared servers, may facilitate access to aggregated third party liability information 1360 for the particular patient. The engine(s) 1315 could facilitate access to the third party liability information 1360 in conjunction with the third party liability information repository.

The user may be provided with an option to view certain payer and/or provider information corresponding to the particular patient. The payer information repository, which could correspond to one or more servers, dedicated or shared in some embodiments, may facilitate access to the aggregated payer information 1350 for the particular patient. The user may wish to view the provider information 1355 and select that option. Responsive to a user selection, the provider information repository, which could correspond to one or more servers, dedicated or shared in some embodiments, may facilitate access to aggregated provider information 1355 for the particular patient. The engine(s) 1315 may facilitate access to the health information 1345 and/or third party liability information 1360 in conjunction with the health information repository and third party liability repository.

The interface may provide an option for the user to view payer information 1350. Responsive to user selection, the interface may provider payer information including, without limitation, payer identification information, payer policy information, payer policy information, coverage/benefits guidelines/rules for services, healthcare plans, explanations of benefits, in-network/preferred provider information, third party payer/obligor information, settlement information, and the like, in general, and/or with respect to the particular patient. The payer information 1350 could indicate qualifying information necessary for determinations of coverage eligibility for healthcare services. Responsive to a user selection, the payer information repository, which in some embodiments could correspond to one or more dedicated or shared servers, may facilitate access to aggregated provider information for the particular patient. The engine(s) 1315 may facilitate access to the health information 1345 in conjunction with the payer information repository.

The interface may provide an option for the user to view third party payer/obligors and/or settlement information which are applicable to the patient and which are eligible for cost-sharing or reimbursement by a healthcare payer. By way of non-limiting example, responsive to a user selection, the interface may present a third party insurance company obligated to pay for a patient's healthcare costs for a specific incident or injury, and/or the extent of coverage from zero-cost sharing to cost-sharing to no coverage. Responsive to user selections, the third party liability information repository, which in some embodiments could correspond to one or more dedicated or shared servers, may facilitate access to the third party liability information 1360 for the particular patient. The engine(s) 1315 may facilitate access to the third party liability information 1360 in conjunction with the third party liability information repository.

With the patient 1260 having already been identified and potential third party payers/obligors been identified, certain categories of payers may have been already eliminated as not being relevant to the patient 1260 at a particular time or for a particular service. For example, a patient 1260 may be eligible for healthcare coverage through Medicare, however a third party may be liable for full coverage of the patient's healthcare costs. Hence, the healthcare provider 1270 need only bill the third party insurer rather than seeking reimbursement from Medicare. Certain categories of payers may be identified as of particular relevance to the patient, such as, but not limited to, those based on a previously retained patient history.

In some embodiments, the user may only see the results of the correlation of the patient's health service costs to third party liability/settlement information. The interface may present billing information, indicating which third party payers/obligors may be liable for the patient's health care coverage and which third party payers/obligors may be applicable to the patient depending on the medical and/or legal judgment of a healthcare provider tending to the patient and/or depending on additional information needed to make the determination. The interface may present options for the user to identify criteria needed for applicability/eligibility, explanation(s) and/or other potentially relevant information. The interface may present options for the user to identify regulatory information pertinent to the obligations of third parties and/or to a legal settlement. The regulatory information may be provided with the third party liability information repository and may include, for example, regulations issued by a government authority, rules/guidelines for implementing regulations, and/or the like.

The interface may provide options for explanations describing coverage eligibility, third party liabilities, reimbursement guidelines that might specify what qualifying information is needed in order to determine what services for which the payer will provide payment and other further information needed, other potentially relevant information, etc. In some embodiments, the interface may provide an option to view a workflow that can be specified for the healthcare provider. The workflow could include a decision tree to gather information used in the determination of third party liability with areas of legal judgment left to the provider. The workflow could include potential questions for the provider's use. The provider may use the solicited information in combination with the provider's judgment and other information identified as potentially relevant by the system to identify, confirm, and/or question whether a particular third party may be liable for a patient's healthcare costs. As an example, a patient may present in the Emergency Department of a hospital with a broken hip. The patient may have hip surgery and a lengthy hospital stay following the surgery. At the time of billing, the hospital may use a system as described herein to consider possibly liable third parties. The system might find a bill from an ambulance company for transporting the patient to the hospital from a grocery store. The system may further find a new court case filed against that grocery store in the patient's name and in the county where the patient resides. That information may be presented to the hospital billing coordinator to inform a decision as to whether to bill the patient's health insurer or continue to investigate whether there is a different third party liable for the patient's healthcare costs associated with the hip injury. The system may present the hospital billing coordinator with the contact information for the attorney who filed the court case and a workflow entry to contact the attorney, with a list of suggested questions to ask the attorney.

The healthcare provider 1270 may provide input via the provider interface 1275. The input may include, for example, corrections to information presented, additional information obtained from the examination of the patient, additional information obtained from speaking with the patient and/or the patient's legal representatives, additional information obtained from speaking with outside parties, etc. In some embodiments, the patient 1260 may provide the input via the patient interface 1265. The input can be similar but confined to appropriate content from a patient's perspective. Pursuant to the input being received, the interface may present an option to regenerate third party liability and/or settlement information, taking the provider's and/or patient's input into account.

Determining third party liability utilizing single or multiple data sources can provide significant financial benefits to healthcare providers, including, but not limited to, reducing repayment costs for governmental and private healthcare insurers, increasing reimbursement rates for hospitals and other healthcare providers (which will allow for better patient care), cost savings to Medicare, Medicaid, other government healthcare programs and private healthcare insurers, and streamlined reimbursement procedures for hospitals and other healthcare providers. Consolidating and error-checking data, for example, using a consolidation engine 1320 and/or a data integrity engine 1330 as part of health information handling system 1220, reduces the number of databases and/or applications that must be consulted for a healthcare provider to collect and view the information necessary to determine whether and when to bill a third party, reducing the number of “clicks” or other inputs required to complete the task, reducing the time required to complete the task, and typically reducing the processing capacity required to run multiple programs as compared to a single program. By storing consolidated files, the communications and processing bandwidth needed to identify, download, and process information from various sources repetitively may also be reduced, as well as the storage capacity (e.g., bytes of computer memory) that would otherwise be required to store all of the relevant information.

From the foregoing, it will be seen that this disclosure is well adapted to attain all the ends and objects hereinabove set forth together with other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth or shown in the accompanying drawings is to be interpreted as illustrative and not in a limiting sense.

What is claimed is:

1. A method for determining the existence of a third party payer/obligor, the method comprising:
accessing a first set of third party liability information for an identified patient, wherein the first set of third party liability information:
is derived from a first electronic data source; and
includes a first indication corresponding to one or more of:
an indication of an existing third party payer/obligor,
an indication of an existing legal or judicial settlement,
an indication of an existing promise or agreement by a third party to reimburse the healthcare provider for all or some part of the identified patient's healthcare costs,
an indication of a first health condition of the identified patient, or
an indication of a first healthcare service provided for the identified patient;
accessing a second set of third party liability information for the identified patient, wherein the second set of third party liability information for the identified patient:
is derived from a second source; and
includes a second indication corresponding to one or more of:
an indication of an existing third party payer/obligor,
an indication of an existing legal or judicial settlement,
an indication of an existing promise or agreement of a third party to reimburse the healthcare provider for all or some part of the identified patient's healthcare costs,
an indication of a first health condition of the identified patient, or
an indication of a first healthcare service provided for the identified patient;
characterizing one or more items from the first set of third party liability information for the identified patient to yield a first set of characterized items;
characterizing one or more items from the second set of third party liability information for the identified patient to yield a second set of characterized items;
determining whether any of the items in the first set of characterized items correspond to any of the items in the second set of characterized items;
checking for errors, inconsistencies, or improbabilities in the values of any items from the first set of characterized items determined to correspond to an item in the second set of characterized items;
resolving any errors, inconsistencies, or improbabilities, or flagging any errors, inconsistencies, or improbabilities that cannot be automatically resolved; and
consolidating the first and second sets of third party information to form a composite set of third party liability information for the identified patient.

2. The method of claim 1, further comprising processing the composite set of third party liability information for the identified patient to identify potential categories of third party liability.

3. The method of claim 2, further comprising processing the composite set of third party liability information for the identified patient to identify potentially liable third party(s).

4. The method of claim 3, further comprising identifying a threshold for assigning third party liability to one or more of the potentially liable third party(s).

5. The method of claim 4, further comprising comparing the threshold for assigning third party liability to one or more of the potentially liable third party(s) to the composite set of third party liability information for the identified patient.

6. The method of claim 5, further comprising flagging a patient or billing file, notifying a healthcare provider, or notifying the patient if the threshold for assigning third party liability to one or more of the potentially liable third party(s) is met.

7. The method of claim 5, wherein if the threshold for assigning third party liability is not met, the sufficiency of information to assign third party liability is assessed.

8. The method of claim 3, wherein processing the composite set of third party liability information comprises selecting an item from the composite set of third party liability information, and assigning the item a weight according to a score for reliability of the information, and identifying potentially liable third party(s) based at least in part on the weighted item.

9. The method of claim 8, wherein the score for reliability is based on one or more of a source of the information, a completeness of the information, or both.

10. The method of claim 9, further comprising scoring a likelihood of third party payment based on the weight assigned to the information used to identify the third party payment.

11. A system for determining the existence of a third party payer/obligor, the system comprising:
one or more interfaces configured to exchange information with at least one of a healthcare provider and a patient;
a network over which information may be exchanged between other system components;
a data consolidation engine configured to:
access a first set of third party liability information for an identified patient;
access a second set of third party liability information for the identified patient;
characterize one or more items from the first set of third party liability information for the identified patient to yield a first set of characterized items;
characterize one or more items from the second set of third party liability information for the identified patient to yield a second set of characterized items;
determine whether any of the items in the first set of characterized items correspond to any of the items in the second set of characterized items; and
consolidate the first and second sets of third party information to form a composite set of third party liability information for the identified patient; and
a data integrity engine configured to check for errors, inconsistencies, or improbabilities in the values of any items from the first set of characterized items determined to correspond to an item in the second set of characterized items and resolve any errors, inconsistencies, or improbabilities or flag any errors, inconsistencies, or improbabilities that cannot be automatically resolved.

12. The system of claim 11, wherein the consolidation engine consolidates the first set of third party liability information and the second set of third party liability information after the data integrity engine has resolved or flagged any errors, inconsistencies, or improbabilities.

13. The system of claim 11, further comprising a third party liability engine configured to derive third party liability/settlement information for the identified patient from one or more information sources in one or more electronic repositories.

14. The system of claim 11, further comprising a delta engine configured to identify changes in third party liability guidelines.

15. The system of claim 14, wherein the delta engine is further configured to determine whether a particular change in third party liability guidelines applies to a particular patient.

16. The system of claim 11, further comprising an information query engine configured to search one or more information repositories electronically accessible by the system.

17. Computer storage media embodying computer-executable instructions which, when executed by one or more processors, cause the one or more processes to perform a method for determining the existence of a third party payer/obligor, the method comprising:
accessing a first set of third party liability information for an identified patient, wherein the first set of third party liability information:
is derived from a first electronic data source; and
includes a first indication corresponding to one or more of:
an indication of an existing third party payer/obligor,
an indication of an existing legal or judicial settlement,
an indication of an existing promise or agreement by a third party to reimburse the healthcare provider for all or some part of the identified patient's healthcare costs,
an indication of a first health condition of the identified patient, or
an indication of a first healthcare service provided for the identified patient;
accessing a second set of third party liability information for the identified patient, wherein the second set of third party liability information for the identified patient:
is derived from a second source; and
includes a second indication corresponding to one or more of:
an indication of an existing third party payer/obligor,
an indication of an existing legal or judicial settlement,
an indication of an existing promise or agreement of a third party to reimburse the healthcare provider for all or some part of the identified patient's healthcare costs,
an indication of a first health condition of the identified patient, or
an indication of a first healthcare service provided for the identified patient;
characterizing one or more items from the first set of third party liability information for the identified patient to yield a first set of characterized items;
characterizing one or more items from the second set of third party liability information for the identified patient to yield a second set of characterized items;
determining whether any of the items in the first set of characterized items correspond to any of the items in the second set of characterized items;
checking for errors, inconsistencies, or improbabilities in the values of any items from the first set of characterized items determined to correspond to an item in the second set of characterized items;
resolving any errors, inconsistencies, or improbabilities, or flagging any errors, inconsistencies, or improbabilities that cannot be automatically resolved; and
consolidating the first and second sets of third party information to form a composite set of third party liability information for the identified patient.

18. The media of claim 17, further comprising processing the composite set of third party liability information for the identified patient to identify potential categories of third party liability.

19. The media of claim 17, further comprising processing the composite set of third party liability information for the identified patient to identify potentially liable third party(s).

20. The media of claim 17, further comprising selecting an item from the composite set of third party liability information, and assigning the item a weight according to a score for reliability of the information, and identifying potentially liable third party(s) based at least in part on the weighted item.
